(19) United States
(12) Patent Application Publication
(10) Pub. No.: US 2008/0228268 A1
(43) Pub. Date: Sep. 18, 2008

(54) Method of Formation of Viscous, Shape Conforming Gels and Their Uses as Medical Prosthesis

(75) Inventors: Kevin F. Shannon, Irving, TX (US); John V. St. John, Grapevine, TX (US); Bill C. Ponder, Colleyville, TX (US)

Correspondence Address: FOLEY & LARDNER LLP, 975 PAGE MILL ROAD, PALO ALTO, CA, 94304, US

(73) Assignee: ULURU, Inc.

(21) Appl. No.: 11/686,902

(22) Filed: Mar. 15, 2007

(51) Int. Cl. A61K 47/30; A61F 2/02; A61K 9/00; B01J 13/14

(52) U.S. Cl. 623/11.11; 424/501; 514/772.3; 516/104; 516/108

(57) ABSTRACT

This invention provides a viscous, shape conforming gel, comprising between about 1% and 50% by weight (dry) of a plurality of polymeric nanoparticles suspended in a liquid or liquids, at least one of which is polar. The plurality of polymeric nanoparticles contained in the gel have an average diameter of less than 1 micrometer and are comprised of an effective amount of polymeric strands each of which is obtained by polymerization of an effective amount of a monomer or two or more monomers in a polar liquid or a mixture of two or more miscible liquids, at least one of which is polar, and an effective amount of a surfactant to stabilize the plurality of gel particles, thereby forming a suspension of gel particles.

### FIELD OF THE INVENTION

This invention relates to the fields of polymer chemistry, physical chemistry, pharmaceutical science, material science and medicine.

### BACKGROUND OF THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

A gel is a three-dimensional polymeric network that has absorbed a liquid to form a stable, usually soft and pliable, composition having a non-zero shear modulus. When the absorbed liquid by a gel is water, the gel is called a hydrogel. Water may comprise a significant weight percent of a hydrogel. This unique characteristic in combination with the fact that many hydrogel-forming polymers are biologically inert, provides opportunities to utilize hydrogels in a wide variety of biomedical applications.

For example, hydrogels are widely used as soft contact lenses. They are also used as burn and wound dressings, with and without incorporated drugs that can be released from the gel matrix to aid in the healing process (e.g., see U.S. Pat. Nos. 3,063,685; 3,963,685 and 4,272,518). Hydrogels have also found utility as devices for the sustained release of biologically active substances. For example, U.S. Pat. No. 5,292,515 discloses a method of preparing a hydrophilic reservoir drug delivery device suitable for mammalian subcutaneous implantation. The '515 patent discloses that the drug release rate can be controlled by the water content of the hydrogel implant, which directly affects its permeability coefficient.

In all of the above patents, the hydrogel is in bulk form, that is, it is an amorphous mass of material with no discernable regular internal structure. Bulk hydrogels have slow swelling rates due to the large internal volume relative to the surface area through which water must be absorbed. Furthermore, a substance dissolved or suspended in the absorbed water will diffuse out of the gel at a rate that depends on the distance it must travel to reach the outer surface of the gel. This situation can be ameliorated to some extent by using particulate gels. If each particle is sufficiently small, substances dispersed in the particles will diffuse to the surface and be released at approximately the same time.

Particulate gels can be formed by a number of procedures as direct or inverse emulsion polymerization (Landfester, et al., (2000) Macromolecules 33:2370) or they can be created from bulk gels by drying the gel and then grinding the resulting xerogel to small particles of a desired size. The particles can then be re-solvated to form particulate gels. Particles having sizes in the micro (10⁻⁶ meters (m)) to nano (10⁻⁹ m)) diameter range can be produced by this means. Molecules of a substance occluded by particles in these size ranges will all have about the same distance to travel to reach the outer surface of the particle and will exhibit in some cases near zero-order release kinetics. However, particulate gels have their own problems. For instance, it is difficult to control the dissemination of the particles to, and localization at, a selected target site. Furthermore, while bulk hydrogels can be rendered shape-retentive, making them useful as biomaterials in a variety of medical applications, currently available particulate gels cannot.

Co-pending U.S. Patent Application Publ. No. U.S. 2004/0086548A1 discloses a shape-retentive aggregate formed from hydrogel particles, thus combining the shape-retentive attributes of bulk hydrogels with the substance release control of particulate gels. This application discloses a method of forming the shape-retentive aggregates by preparing a suspension of hydrogel particles in water and concentrating the suspension until the particles coalesce into a shape-retentive aggregate held together by non-covalent bond forces including but not limited to hydrophobic/hydrophilic interactions and hydrogen bonding.

Co-pending U.S. Patent Application Publ. No. U.S. 2005/0118270A1 discloses a method of forming shape-retentive aggregates in situ, such that the shape of the aggregate would be dictated by the shape of the locus of application. Aggregate formation is accomplished by introducing a suspension of gel particles dispersed in a polar liquid, wherein the gel particles have an absolute zeta potential enabling the particles to remain dispersed, into a receiving medium wherein the absolute zeta potential of the gel particles is reduced. The gel particles coalesce into a shape-retentive aggregate held together by non-covalent bond physical forces comprising hydrophobic-hydrophilic interactions and hydrogen bonding.

Reconstructive surgery has been used for many years for the treatment of congenital tissue defects, for repair of damaged organs and tissues and for tissue augmentation. An ideal material for mammalian tissue reconstruction should be biocompatible, able to incorporate into the native tissue without inducing an adverse tissue response, and should have adequate anatomical and functional properties (for example, size, strength, durability, and the like). Although a large number of bio-materials, including synthetic and naturally-derived polymers, have been employed for mammalian tissue reconstruction or augmentation (see, e.g., “Textbook of Tissue Engineering” Eds. Lanza, R., Langer, R., and Chick, W., ACM Press, Colorado (1996) and references cited therein), no material has proven satisfactory for use in every application.

### DETAILED DESCRIPTION OF THE INVENTION

This invention provides a hydrogel composition that is particularly useful in many commercial applications where the locus of application is in vivo, e.g., biomedical applications such as joint reconstruction and cosmetic surgery.

In one aspect this invention provides a viscous, shape conforming gel, comprising between about 1% and 50% by weight (dry) of a plurality of polymeric nanoparticles suspended in a liquid, at least one of which is polar. The plurality of polymeric nanoparticles have an average diameter of less than 1 micrometer and are comprised of an effective amount of polymeric strands each of which is obtained by polymerization of an effective amount of a monomer or two or more monomers, at least one of which is a 2-alkenoic acid, a hydroxy (2C-4C) alkyl 2-alkenoate, a hydroxy (2C-4C) alkoxy (2C-4C) alkyl 2-alkenoate, a dihydroxy (2C-4C) alkyl 2-alkenoate , a (1C-4C) alkoxy (2C-4C) alkoxy (2C-4C) alkyl 2-alkenoate or a vicinyl epoxy (1C-4C) alkyl 2-alkenoate, in an effective amount of a liquid, at least one of which is polar, or an effective amount of a mixture of two or more miscible liquids, at least one of which is polar, and an effective amount of a surfactant to stabilize the plurality of gel particles. The effective amounts of the above components in the gel suspension or system are provided such that the naonoparticles are at a concentration of from about 300 to about 1200 mg wet weight/mL in the suspension system. In one aspect, the amount of powdered nanoparticles is from about 1% to about 50% by weight (dry), or in an alternate embodiment, is about 2% to about 30% by weight (dry) or yet further, is about 8% to about 20% by weight (dry).

Thus, the present invention provides a suspension made from a dry powder of polymeric nanoparticles. The nanoparticles are suspended in a solvent, at least one of which is polar, the nanoparticles being prepared by polymerizing an effective amount of a monomer or two or more monomers, at least one of which is a 2-alkenoic acid, a hydroxy (2C-4C) alkyl 2-alkenoate, a dihydroxy (2C-4C) alkyl 2-alkenoate, a hydroxy (2C-4C) alkoxy (2C-4C) alkyl 2-alkenoate, a (1C-4C) alkoxy (2C-4C) alkoxy (2C-4C) alkyl 2-alkenoate or a vicinyl epoxy (1C-4C) alkyl 2-alkenoate, in a polar liquid or a mixture of two or more miscible liquids, at least one of which is polar, and an effective amount of a surfactant to produce a suspension of a plurality of polymeric nanoparticles wherein the polymeric nanoparticles have an average diameter of less than 1×10⁻⁶ m; and then removing the liquid(s) from the suspension such that the amount of liquid(s) remaining in the dry powder is less than 10% by weight wherein the percentage is based on the total weight of the dry powder. In one aspect, the amount of powdered nanoparticles is from about 1% to about 50% by weight (dry), or in an alternate embodiment, is about 2% to about 30% by weight (dry) or yet further, is about 8% to about 20% by weight (dry).

This invention also provides a method of forming a viscous, shape conforming suspension of gel particles by reconstituting a dry powder of polymeric nanoparticles. The nanoparticles are prepared as noted above, i.e., by polymerizing an effective amount of a plurality of gel particles having an average diameter of less than 1 micrometer, wherein the gel particles individually comprise an effective amount of a plurality of polymeric strands obtained by polymerization of an effective amount of a monomer or two or more monomers, at least one of which is a 2-alkenoic acid, a hydroxy (2C-4C) alkyl 2-alkenoate, a dihydroxy (2C-4C) alkyl 2-alkenoate, a hydroxy (2C-4C) alkoxy (2C-4C) alkyl 2-alkenoate, a (1C-4C) alkoxy (2C-4C) alkoxy (2C-4C) alkyl 2-alkenoate or a vicinyl epoxy (1C-4C) alkyl 2-alkenoate, in a polar liquid or a mixture of two or more miscible liquids, at least one of which is polar, and an effective amount of a surfactant to stabilize the plurality of gel particles. The effective amounts of the above components are provided such that the gel particles are a concentration at from about 300 to about 1200 mg wet weight/mL in the suspension system. In one aspect, the amount of powdered nanoparticles is from about 1% to about 50% by weight (dry), or in an alternate embodiment, is about 2% to about 30% by weight (dry) or yet further, is about 8% to about 20% by weight (dry).

One embodiment of this invention does not include compositions comprising a homopolymer poly(2-sulfoethyl methacrylate) (pSEMA).

In another embodiment, a medical prosthesis for tissue reconstruction is provided. The prosthesis is reconstituted from lyophilized gel nanoparticles and comprise a viscous, shape conforming gel containing a plurality of gel particles each having an average diameter of less than 1 micrometer, wherein the gel particles individually comprise an effective amount of a plurality of polymeric strands obtained by polymerization of an effective amount of a monomer or two or more monomers, at least one of which is a 2-alkenoic acid, a hydroxy (2C-4C) alkyl 2-alkenoate, a dihydroxy (2C-4C) alkyl 2-alkenoate, a hydroxy (2C-4C) alkoxy (2C-4C) alkyl 2-alkenoate, a (1C-4C) alkoxy (2C-4C) alkoxy (2C-4C) alkyl 2-alkenoate or a vicinyl epoxy (1C-4C) alkyl 2-alkenoate, in an effective amount of a polar liquid or an effective amount of a mixture of two or more miscible liquids, at least one of which is polar, and an effective amount of a surfactant to stabilize the plurality of gel particles. The effective amounts of the above components are provided such that the gel particles are at a concentration of from about 300 to about 1200 mg wet weight/mL in the suspension system. In one aspect, the amount of powdered nanoparticles is from about 1% to about 50% by weight (dry), or in an alternate embodiment, is about 2% to about 30% by weight (dry) or yet further, is about 8% to about 20% by weight (dry).

The compositions and prosthesis of this invention are useful in tissue reconstruction. This invention also provides these methods for their use in tissue reconstruction as well.

### BRIEF DESCRIPTION OF TABLES AND FIGURES

Table 1 shows the nanoparticle size before and after lyophilization for pHEMA, pHPMA and copolymers of pHEMA:HPMA

Table 2 shows the relative masses and mmol of monomers in preparation of cross-linked nanoparticles composed of copolymers of HPMA and Methacrylic acid (MAA).

Table 3 shows the average size and particle size range for cross-linked nanoparticles composed of copolymers of HPMA and Methacrylic acid (MAA).

Table 4 shows the relative masses and mmol of monomers in Preparation of cross-linked nanoparticles composed of copolymers of HEMA and GMA.

Table 5 shows the average size and particle size range for cross-linked nanoparticles composed of copolymers of HEMA and GMA.

Table 6 shows the viscosity for gels with the same polymer concentration but different chemical compositions.

Table 7 shows the relative amount of deformation in gels of different compositions at the same polymer concentration utilizing a 10 gram weight.

FIG. 1 is a photograph showing the hydrogel nanoparticle powder, the powder applied to phosphate buffered saline and the resulting aggregate film after the powder hydrates.

FIG. 2 is an image showing the nanoparticle suspension, nanoparticle powder, viscous gel, and resulting nanoparticle aggregate after exposure to physiological saline.

FIG. 3 is a plot showing the change in nanoparticle size with increasing concentration as a gel when nanoparticles are redispersed after gel formation.

FIG. 4 is a plot showing the change in viscosity of gels as the concentration of nanoparticles is increased.

FIG. 5 is a plot showing the change in viscosity over time for gels with different concentration of dry polymer nanoparticles.

FIG. 6 is a plot showing the change in relative deformation of gels with increasing polymer concentration using a 10 gram weight.

FIG. 7 is a plot showing the relative rate of aggregation for viscous gels composed of different compositions of nanoparticles.

FIG. 8 is a plot showing the relative deflection for viscous gels composed of different nanoparticle compositions.

FIG. 9 is a plot showing relative deflection for viscous gels composed of different percentages of polymer dispersions in water.

FIG. 10 shows the effect on the viscous gel contained within an implant surgically implanted in a rabbit after rupturing the shell.

### MODES FOR CARRYING OUT THE INVENTION

Definitions

As used herein, the term “comprising” is intended to mean that the compositions and methods include the recited elements, but not excluding others. “Consisting essentially of” when used to define compositions and methods, shall mean excluding other elements or method steps of any essential significance to the composition or method. For example, a composition consisting essentially of the elements as defined herein would not exclude trace contaminants. “Consisting of” shall mean excluding more than trace elements of other ingredients and substantial method steps. Embodiments defined by each of these transition terms are within the scope of this invention.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art and can be substituted herein.

As used herein, the term “gel” refers to a three-dimensional polymeric structure that itself is insoluble in a particular liquid but which is capable of absorbing and retaining large quantities of the liquid to form a stable, often soft and pliable, but always to one degree or another shape-retentive, structure. When the liquid is water, the gel is referred to as a hydrogel. Unless expressly stated otherwise, the term “gel” will be used throughout this application to refer both to polymeric structures that have absorbed a liquid other than water and to polymeric structures that have absorbed water, it being readily apparent to those skilled in the art from the context whether the polymeric structure is simply a “gel” or a “hydrogel.”

The term “polar liquid,” as used herein has the meaning generally understood by those skilled in the chemical art. In brief, a polar liquid is one in which the electrons are unevenly distributed among the atoms of its molecules and therefore create an electrical dipole. To be polar a molecule must contain at least one atom that is more electronegative than other atoms in the molecule. Examples of polar liquids include, without limitation, water, where the oxygen atom bears a partial negative charge and the hydrogen atoms a partial positive charge, and alcohols, wherein the O—H moiety is similarly polarized.

As used herein, “gel particle” refers to a microscopic or sub-microscopic quantity of a gel in a discrete shape, usually, but not necessarily, spherical or substantially so. The term also intends small clusters of individual particles held together by non-covalent bond physical forces such as hydrophilic/hydrophobic interactions and hydrogen bonding, wherein the clusters do not adversely affect the stability of a gel particle suspension (suspension system) containing them or the performance of the suspension system in the methods of this invention. Clusters result from changes in concentration of gel particles in suspension. That is, at higher concentrations, it is more likely individual particles will get close enough to one another for non-covalent bond forces, to cause them to coalesce unless a sufficient amount of surfactant is present to stabilize a high concentration of gel particles.

As used herein, a “suspension” refers to a uniformly distributed, stable dispersion of a solid in a liquid in which the solid is not soluble. A surfactant is added to the liquid to help stabilize the dispersion. As used herein, a “suspension system” refers to a suspension wherein gel particles of this invention are the dispersed solid. By “stable” is meant that the solid remains uniformly dispersed for at least 24 hours, unless subjected to disrupting external forces such as, without limitation, centrifugation or filtration.

As used herein, a “surfactant” has the meaning generally understood by those skilled in the chemical art. That is, a surfactant is a soluble compound, which may be anionic, cationic, zwitterionic, amphoteric or neutral in charge, and which reduces the surface tension of the liquid in which it is dissolved or that reduces interfacial tension between two liquids or a liquid and a solid.

As used herein, a “viscous, shape conforming gel” refers to a high concentration of gel particles in a polar liquid comprising a surfactant to prevent self-aggregation.

As used herein, a “medically acceptable envelope” means a Food & Drug Administration (FDA) approved material that is currently used to contain silicone, saline or other material for use as a tissue reconstruction implant for use in clinically relevant animal models or human patients.

As used herein, the term “aggregate formation” refers to a process in which the medically acceptable envelope is breached and the gel particles are exposed to a physiological environment, causing a reduction of the absolute zeta potential on the particles and which makes them coalesce into a localized structure composed of a large number of gel particles held together by inter-particle and particle-liquid forces such as, without limitation, hydrophilic/hydrophobic interactions and hydrogen bonding.

As used herein, a “monomer” has the meaning understood by those skilled in the chemical art. That is, a monomer is a small chemical compound that is capable of forming a macromolecule of repeating units of itself, i.e., a polymer. Two or more different monomers may react to form a polymer in which each of the monomers is repeated numerous times, the polymer being referred to as a copolymer to reflect the fact that it is made up of more than one monomer.

As used herein, the term “size” when used to describe a gel particle of this invention refers to the volume of an essentially spherical particle as represented by its diameter, which of course is directed related to its volume. When referring to a plurality of gel particles, size relates to the average volume of the particles in the plurality as represented by their average diameter.

As used herein, the term “polydispersivity” refers to the range of sizes of the particles in a suspension system. “Narrow polydispersivity” refers to a suspension system in which the size of the individual particles, as represented by their diameters, deviates 10% or less from the average diameter of the particles in the system. If two or more pluralities of particles in a suspension system are both stated to be of narrow polydispersivity, what is meant is that there are two distinct sets of particles wherein the particles of each set vary in diameter by no more than 10% from an average diameter of the particles in that set and the two averages are distinctly different. A non-limiting example of such a suspension system would be one comprising a first set of particles in which each particle has a diameter of 20 nm±10% and a second set of particles in which each particle has a diameter of 40 nm±10%.

As used herein, the term “broad polydispersivity” refers to a suspension system in which the size of the individual particles of a set of particles deviates more than 10% from the average size of the particles of the set.

As used herein, the term “plurality” simply refers to more than one, i.e., two or more.

As used herein, the term “chemical composition” as it relates to a gel particle of this invention refers to the chemical composition of the monomers that are polymerized to provide the polymer strands of the particle, to the chemical composition and ratios of different monomers if two or more monomers are used to prepare the polymer strands of the particles and/or to the chemical composition and quantity of any cross-linking agent(s) that are used to inter-connect the particle strands.

As used herein, a “particle strand” refers to a single polymer molecule or, if the system in which the strand exists contains a cross-linking agent, two or more inter-connected polymer molecules. The average number of polymer strands that will be cross-linked and the average number of cross-links between any two polymer strands in a particular gel particle will depend on the quantity of cross-linker in the system and on the concentration of polymer strands.

As used herein, the term “wet weight” refers to the weight of a gel particle after it has absorbed the maximum amount of a liquid it is capable of absorbing. When it is stated that a particle has occluded from about 0.1 to about 99 weight percent of a pharmaceutically active agent-containing liquid, what is meant is that the pharmaceutically active agent-containing liquid makes up from about 0.1 to about 99% of the weight of the particle after occlusion of the pharmaceutically active agent-containing liquid.

As used herein the term “dry weight” means the weight of nanoparticles without the weight of any polar liquid(s).

As used herein, the term “pharmaceutically active agent” refers to any substance that is occluded by a gel particle or is dissolved or dispersed in the polar liquid(s) comprising the viscous shape conforming gel. Examples of pharmaceutically active agents, without limitation, include biomedical agents; biologically active substances such as antibiotics, anti-rejection agents such as immunosuppressive or tolerance-inducing agents, genes, proteins, growth factors, monoclonal antibodies, fragmented antibodies, antigens, polypeptides, DNA, RNA, ribozymes, radiopaque substances and radioactive substances.

As used herein, the term “pharmaceutical active agent” refers to both small molecule and to macromolecular compounds used as drugs. Among the former are, without limitation, antibiotics, chemotherapeutics (in particular platinum compounds and taxol and its derivatives), analgesics, antidepressants, antibiotics, antimicrobials, anti-allergenics, anti-rejection agents such as immunosuppressive or tolerance-inducing agents, anti-arryhthics, anti-inflammatory compounds, CNS stimulants, sedatives, anti-cholinergics, anti-arteriosclerotics, and the like. Macromolecular compounds include, without limitation, monoclonal antibodies (mAbs), Fabs, proteins, peptides, cells, antigens, nucleic acids, genes, proteins, growth factors, antigens, polypeptides, DNA, RNA, ribozymes enzymes, growth factors and the like. A pharmaceutical agent may be intended for topical or systemic use.

As used herein, “hydroxy” refers to an —OH group.

As used herein, the term “alkyl” refers to a straight or branched chain saturated aliphatic hydrocarbon, i.e., a compound consisting of carbon and hydrogen only. The size of an alkyl in terms of how many carbon atoms it contains is indicated by the formula (“a”C-“b”C)alkyl where a and b are integers. For example, a (1C-4C)alkyl refers to a straight or branched chain alkyl consisting of 1, 2, 3, 4 or more carbon atoms. An alkyl group may be substituted or unsubstituted.

As used herein, the term “alkoxy” refers to the group —O-alkyl wherein alkyl is as defined herein. The size of an alkoxy in terms of how many carbon atoms it contains is indicated by the formula (“a”C-“b”C) alkoxy where a and b are integers. For example, a (1C-4C) alkoxy refers to a straight or branched chain —O-alkyl consisting of 1, 2, 3, 4 or more, carbon atoms. An alkoxy group may be substituted or unsubstituted.

As used herein, “ester” refers to the group —C(O)O-alkyl wherein alkyl is as defined herein.

As used herein, “2-alkenoic acid” refers to the group (R¹)(R²)C═C(R³)—C(O)OH wherein each of R¹, R², R³ are independently selected from hydrogen and alkyl wherein alkyl is as defined herein. These 2-alkenoic acids are exemplified, for example by, acrylic acid, methacrylic acid, etc.

As used herein, “2-alkenoate” refers to the group (R¹)(R²)C═C(R³)—C(O)O-alkyl wherein each of R¹, R², R³ are independently selected from hydrogen and alkyl wherein alkyl is as defined herein.

As used herein, the term “cross-linking agent” refers to a di-, tri-, or tetra-functional chemical entity that is capable of forming covalent bonds with functional groups on polymeric strands resulting in a three-dimensional structure.

As used herein, the term “hydrogen bond” refers to the electronic attraction between a hydrogen atom covalently bonded to a highly electronegative atom and another electronegative atom having at least one lone pair of electrons. The strength of a hydrogen bond, about 23 kJ (kilojoules) mol⁻¹, is between that of a covalent bond, about 500 kJ mol⁻¹, and a van der Waals attraction, about 1.3 kJ mold⁻¹. Hydrogen bonds have a marked effect on the physical characteristics of a composition capable of forming them. For example, ethanol has a hydrogen atom covalently bonded to an oxygen atom, which also has a pair of unshared (i.e., a “lone pair”) electrons and, therefore, ethanol is capable of hydrogen bonding with itself. Ethanol has a boiling point of 78° C. In general, compounds of similar molecular weight are expected to have similar boiling points. However, dimethyl ether, which has exactly the same molecular weight as ethanol but which is not capable of hydrogen bonding between molecules of itself, has a boiling point of −24° C., almost 100 degrees lower than ethanol. Hydrogen bonding between the ethanol molecules has made ethanol act as if it were substantially higher in molecular weight.

As used herein, a “charged” gel particle refers to a particle that has a localized positive or negative charge due to ionic content of the monomers making up the polymer strands of the particle and the environment in which these particles find themselves. For example, without limitation, hydrogel particles comprising acrylic acid as a co-monomer will, under basic conditions, exist in a state in which some or all of the acid groups are ionized, i.e., —COOH becomes-COO⁻. Another example is the amino (—NH₂) group, which, in an acidic environment, will form an ammonium (—NH₃⁺) ion.

As used herein, “zeta potential” as used herein has the meaning generally understood by those skilled in the chemical art. Briefly, when a charged particle is suspended in an electrolytic solution, a layer of counter-ions (ions of charge opposite that of the particle) forms at the surface of the particle. This layer of particles is strongly adhered to the surface of the particle and is referred to as the Stern layer. A second, diffuse layer of ions of the same charge as the particle (and opposite the charge of the counter-ions that form the Stern layer, often referred to as co-ions) then forms around the strongly absorbed inner layer. The attached counter-ions in the Stem layer and the charged atmosphere in the diffuse layer are referred to as the “double layer”, the thickness of which depends on the type and concentration of ions in solution. The double layer forms to neutralize the charge of the particle. This causes an electrokinetic potential between the surface of the particle and any point in the suspending liquid. The voltage difference, which is on the order of millivolts (mV) is referred to as the surface potential. The potential drops off essentially linearly in the Stern layer and then exponentially in the diffuse layer.

A charged particle will move with a fixed velocity in a voltage field, a phenomenon that is called electrophoresis. Its mobility is proportional to the electrical potential at the boundary between the moving particle and the surrounding liquid. Since the Stern layer is tightly bound to the particle and the diffuse layer is not, the preceding boundary is usually defined as being the boundary between the Stern layer and the diffuse layer, often referred to as the slip plane. The electrical potential at the junction of the Stern layer and the diffuse layer is related to the mobility of the particle. While the potential at the slip plane is an intermediate value, its ease of measurement by, without limitation, electrophoresis and it direct relationship with stability renders it an ideal characterizing feature of the dispersed particles in suspension. It is this potential that is called the zeta potential. The zeta potential can be positive or negative depending on the initial charge on the particle. The term “absolute zeta potential” refers to the zeta potential of a particle absent the charge sign. That is, actual zeta potentials of, for example, +20 mV and −20 mV would both have an absolute zeta potential of 20.

Charged particles suspended in a liquid tend to remain stably dispersed or to agglomerate depending primarily on the balance between two opposing forces, electrostatic repulsion, which favors a stable dispersion, and van der Waals attraction, which favors particle coalescence or “flocculation” as it is sometimes referred to when the particles initially come together. The zeta potential of the dispersed particles is related to the strength of the electrostatic repulsion so a large absolute zeta potential favors a stable suspension. Thus, particles with an absolute zeta potential equal to or greater than about 30 mV tend to form stable dispersions, since at this level the electrostatic repulsion is sufficient to keep the particles apart. On the other hand, when the absolute value of the zeta potential is less than about 30, then van der Walls forces are sufficiently strong to overcome electrostatic repulsion and the particles tend to flocculate.

The zeta potential of a particle of a particular composition in a particular solvent may be manipulated by modifying, without limitation, the pH of the liquid, the temperature of the liquid, the ionic strength of the liquid, the types of ions in solution in the liquid, and the presence, and if present, the type and concentration of surfactant(s) in the liquid.

As used herein, an “excipient” refers to an inert substance added to a pharmaceutical composition to facilitate its administration. Examples, without limitation, of excipients include calcium carbonate, calcium phosphate, various sugars and types of starch, cellulose derivatives, gelatin, vegetable oils and polyethylene glycols. A “pharmaceutically acceptable excipient” refers to an excipient that does not cause significant irritation to an organism and does not abrogate the biological activity and properties of the administered compound.

The viscous, shape conforming gels of this invention may be manipulated using the disclosures herein so as to be capable of occluding and/or entrapping virtually any pharmaceutical agent presently known, or that may become known, to those skilled in the art as being effective in the treatment and/or prevention of any of the above diseases and all such pharmaceutical agents are within the scope of this invention.

As used herein, the term “in vivo” refers to any process or procedure performed within a living organism, which may be a plant or an animal, in particular, in a human being.

As used herein, the term “hydrophilic/hydrophobic interactions” refers to the inter-or intra-molecular association of chemical entities through physical forces, whereby hydrophilic compounds or hydrophilic regions of compounds tend to associate with other hydrophilic compounds or hydrophilic regions of compounds, and hydrophobic compounds or hydrophobic regions of compounds tend to associate with other hydrophobic compounds or hydrophobic regions of compounds.

As used herein, the term “occlude” has the meaning generally understood by those skilled in the chemical art, that is, to absorb and retain a substance for a period of time. With regard to this invention, substances may be absorbed by and retained in, i.e. occluded by, gel particles of this invention during their formation.

As used herein, the term “entrapped” refers to the retention for a period of time of a substance in the voids between the gel particles comprising the viscous, shape conforming gel of this invention.

As used herein, the term “average molecular weight” refers to the weight of individual polymer strands or cross-linked polymer strands of this invention. For the purpose of this invention, average molecular weight is determined by gel permeation chromatography with laser light scattering detection.

As used herein, the term “elastic modulus” refers the stiffness of a given material, and is the ratio of linear stress in a body to the corresponding linear strain within the limits of elasticity.

A used herein, the term “viscoelastic” refers to a material that exhibits both viscous and elastic properties, that is a material will deform and flow under the influence of an applied shear stress, but will slowly recover from some of the deformation.

As used herein, the term “self-aggregation” refers to the process by which gel particles, due to their close proximity to each other in concentrated suspensions, coalesce and form a solid mass regardless of the type and amount of surfactant present.

As used herein, the term “self sealing” refers to the process in which the gel particles aggregate at the implant rupture site, preventing additional material from exiting the shell.

A “composition” is intended to mean a combination of the suspension or other agent and another compound or composition, or carrier, e.g., a liquid carrier inert or active, such as a therapeutic.

A “pharmaceutical composition” is intended to include the combination of the an active pharmaceutical with a carrier such as the suspension of this invention, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin REMINGTON'S PHARM. SCI., 15th Ed. (Mack Publ. Co., Easton (1975)).

An “effective amount” is an amount sufficient to effect beneficial or desired results. Methods for determining the effective amount, as determined by the desired or beneficial result, are known in the art.

### Embodiments

This invention provides a viscoelastic gel comprised of a dry powder of polymeric nanoparticles suspended in at least one polar liquid. Methods of making the suspension as well as use thereof are provided as well.

An aspect of the hydrogel nanoparticle viscoelastic gel is its concentration and thus in one aspect, an object of this invention is to produce a suspension of gel particles at a high concentration, in order to minimize the injection volume when introduced in vivo to form an aggregate with the desired physical properties for a specific application.

Another objective is to prevent the gel particles in suspension from self-aggregating without introducing the suspension into an environment that causes the particles to aggregate due to a reduction in absolute zeta potential. This is accomplished by utilizing appropriate pharmaceutically accepted surfactants at specific concentrations that stabilize these high concentrations of particles. This can be accomplished by a determined ratio between concentration of gel particles and type and amount of surfactant necessary to prevent self-aggregation.

For each specific commercial application, it is apparent that different concentrations of both gel particles and surfactants may be required. In determining the relationship between gel concentration and surfactant level, hydrogel nanoparticles were isolated by several methods, one of which was lyophilization. The dry, hydrogel particles were then resuspended in the presence of a surfactant to determine the maximum concentration that could be attained without aggregation occurring.

During these specific experiments, it was discovered that as the concentration increased beyond the 300 mg/mL net weight or an alternate embodiment, greater than 500 mg/mL net weight, and at a fixed level of surfactant, the suspensions did not aggregate and in fact were forming viscoelastic gels with different physical properties than those of true aggregates. These viscous gels varied in viscosity depending upon the concentration of the dispersed nanoparticles. The viscous gels showed no retention of shape as a true nanoparticle aggregate behaves. The material physical properties of these viscous gels could be altered from a honey consistency at lower viscosity to a rubber type of material at high concentration and viscosity. The higher viscosity gels were of most interest, since the viscoelastic properties were approaching those of soft tissue, including tissue containing adipose tissue. None of these materials behave as a shape-retentive aggregates but rather a flowing, amorphous liquid with high viscosity and take the shape, which when contained within an envelope will take the shape of a container. However, as expected, the viscous gels would aggregate if the absolute zeta potential on the particles comprising these viscous gels was reduced, e.g., by exposing them to a physiological environment. It was therefore unexpected that a new, safe, unique medical prosthesis for mammalian tissue reconstruction, utilizing a maximum concentration of gel particles suspended in water or other polar solvent with a sufficient amount of surfactant to prevent self-aggregation would result.

In one aspect, the gel particles suspended in a polar solvent, preferable water, and in the presence of a pharmaceutically acceptable surfactant are introduced into a suitable, medically acceptable implantable, water impermeable envelope, composed of, for example, silicone elastomer or polyurethane, and the properties of the resulting implant such as softness and elastic modulus can be easily adjusted by the composition and amount of hydrogel nanoparticles and surfactant concentration. Another advantage is that if a rupture or catastrophic failure would occur, the leakage would be localized and the viscous gel particles would form a biologically safe, localized aggregate that could be surgically removed. An additional advantage, utilizing the drug delivery capabilities of the hydrogel nanoparticle chemistry the suspensions can further contain pharmaceuticals or other agents, e.g., antibiotics and anti-rejection agents, within the viscous gels or occluded inside the gel particles comprising the viscous gels. Utilizing a medically acceptable implantable envelope that is permeable to certain drugs to contain the viscous gel, the implant could provide a sustained, localized delivery of the active through the envelope into the surrounding tissue. With the major problems and limitations of current mammalian tissue reconstruction implants with respect to rejection, infection, leakage of toxic liquid if ruptured, and “feel”, these additional attributes provide a technology base for numerous medical applications.

The suspension is prepared from a dry powder of polymeric nanoparticles. The dry powder is prepared by polymerizing an effective amount of a monomer or two or more monomers, at least one of which is a 2-alkenoic acid, a hydroxy (2C-4C) alkyl 2-alkenoate, a dihydroxy (2C-4C) alkyl 2-alkenoate, a hydroxy (2C-4C) alkoxy (2C-4C) alkyl 2-alkenoate, a (1C-4C) alkoxy (2C-4C) alkoxy (2C-4C) alkyl 2-alkenoate or a vicinyl epoxy (1C-4C) alkyl 2-alkenoate, in a polar liquid or a mixture of two or more miscible liquids, at least one of which is polar, and an effective amount of a surfactant to produce a suspension of a plurality of polymeric nanoparticles wherein the polymeric nanoparticles have an average diameter of less than 1×10⁻⁶ m. After polymerization, the liquid(s) are removed from the suspension such that the amount of liquid(s) remaining in the dry powder is less than 10% by weight wherein the percentage is based on the total weight of the dry powder. Alternate embodiments of the varying polymer combinations and liquids are described herein.

In one aspect, this invention provides a method of forming a viscous, shape conforming suspension of gel particles by dispersing a lyophilized concentrated plurality of gel particles having an average diameter of less than 1 micrometer, wherein the gel particles comprise an effective amount of a plurality of polymeric strands obtained by polymerization of an effective amount of a monomer or two or more monomers, at least one of which is a 2-alkenoic acid, a hydroxy (2C-4C) alkyl 2-alkenoate, a dihydroxy (2C-4C) alkyl 2-alkenoate, a hydroxy (2C-4C) alkoxy (2C-4C) alkyl 2-alkenoate, a (1C-4C) alkoxy (2C-4C) alkoxy (2C-4C) alkyl 2-alkenoate or a vicinyl epoxy (1C-4C) alkyl 2-alkenoate, in an effective amount of a polar liquid or a mixture of two or more miscible liquids, at least one of which is polar, and an effective amount of a surfactant to stabilize the plurality of gel particles, thereby forming a suspension of gel particles wherein the particles are concentrated at from about 300 to about 1200 mg wet weight/mL in the suspension system. In alternative embodiments, the particles in the suspension system are concentrated at from about 300 to about 1000 mg wet weight/mL, or alternatively from about 300 to about 800 mg wet weight/mL, or alternatively from about 300 to about 600 mg wet weight/mL, or alternatively from about 500 to about 1200 mg wet weight/mL, or alternatively from about 700 to about 1200 mg wet weight/mL, or alternatively from about 900 to about 1200 mg wet weight/mL, or alternatively from about 500 to about 1000 mg wet weight/mL, or yet further, greater than 300 mg wet weight/mL or yet further, greater than 500 mg wet weight/mL. In a further aspect, the amount of particles can be defined by the percentage of nanoparticles by weight (dry). In one aspect, the amount of powdered nanoparticles is from about 1% to about 50% by weight (dry), or in an alternate embodiment, is about 2% to about 30% by weight (dry) or yet further, is about 8% to about 20% by weight (dry).

In another embodiment, the at least one monomer is acrylic acid, methacrylic acid, 2-hydroxyethyl acrylate, 2-hydroxyethylmethacrylate, diethyleneglycol monoacrylate, diethyleneglycol monomethacrylate, 2-hydroxypropyl acrylate, 2-hydroxypropyl methacrylate, 3-hydroxypropyl acrylate, 3-hydroxypropyl methacrylate, dipropylene glycol monoacrylate, dipropylene glycol monomethacrylate, gylcidyl methacrylate, 2,3-dihydroxypropyl methacrylate, or glycidyl acrylate.

In another embodiment, the monomer(s) is/are 2-hydroxyethyl methacrylate, 2-hydroxypropyl methacrylate, 3-hydroxypropyl methacrylate, glycerol methacrylate, or a combination thereof. In a further embodiment, only one polymer type is used such as 2-hydroxyethyl methacrylate 2-hydroxypropyl methacrylate, 3-hydroxypropyl methacrylate or 2,3-dihydroxypropyl methacrylate. In another aspect, the polymer is a combination of two polymer types, one of which is 2-hydroxyetheyl methacrylate, 2-hydroxypropyl methacrylate, 3-hydroxypropyl methacrylate or 2,3-dihydroxypropyl methacrylate.

In another embodiment, the gel particles are about the same average diameter, are formed from one or more monomers and are of a narrow polydispersivity. In another embodiment, the gel particles are of differing average diameter, are formed from one or more monomers and are of a narrow polydispersivity.

In another embodiment, the gel particles are formed from one or more monomers and are of a broad polydispersivity.

In another embodiment, the plurality of gel particles in the suspension system is at a concentration in the range of about 5-20% that results in cluster formation. In alternative embodiments, the plurality of gel particles in the suspension system is at a concentration in the range of about 5-10%, or alternatively about 5-15%, or alternatively about 10-20%, or alternatively about 15-20%, or alternatively about 10-15%, or alternatively about 6-19%, or alternatively about 7-18% that results in cluster formation.

In another embodiment, the effective amount of the surfactant is from about 0.005 weight percent to about 0.50 weight percent. In alternative embodiments, the effective amount of the surfactant is from about 0.005 weight percent to about 0.1 weight percent, or alternatively from about 0.005 weight percent to about 0.2 weight percent, or alternatively from about 0.005 weight percent to about 0.3 weight percent, or alternatively from about 0.005 weight percent to about 0.4 weight percent, or alternatively from about 0.05 weight percent to about 0.1 weight percent, or alternatively from about 0.05 weight percent to about 0.2 weight percent, or alternatively from about 0.05 weight percent to about 0.3 weight percent, or alternatively from about 0.05 weight percent to about 0.4 weight percent, or alternatively from about 0.05 weight percent to about 0.5 weight percent, or alternatively from about 0.006 weight percent to about 0.40 weight percent.

In another embodiment, the average diameter of the gel particles is from about 1 to about 1,000 nanometers. In alternative embodiments, the average diameter of the gel particles is from about 10 to about 1,000 nanometers, or, or alternatively from about 100 to about 1,000 nanometers, or alternatively from about 10 to about 100 nanometers, or alternatively from about 20 to about 1,000 nanometers.

In another embodiment, the average diameter of the gel particles is from about 40 to about 800 nanometers. In alternative embodiments, the average diameter of the gel particles is from about 40 to about 500 nanometers, or alternatively from about 40 to about 300 nanometers, or alternatively from about 100 to about 800 nanometers, or alternatively from about 300 to about 800 nanometers, or alternatively from about 600 to about 800 nanometers, or alternatively from about 50 to about 700 nanometers. In a yet further embodiments, the average diameter of the gel particles is greater than about 35 nanometers, or yet further 55 nanometers, or yet further greater than about 75 nanometers, or yet further greater than about 100 nanometers, or yet further greater than about 150 nanometers, or yet further greater than about 200 nanometers, or yet further greater than about 250 nanometers, 300 nanometers, or yet further greater than about 350 nanometers, or yet further greater than about 400 nanometers.

In another embodiment, the gel particles are at a concentration of from about 500 to about 900 mg wet weight/mL in the suspension system. In alternative embodiments, the gel particles in the suspension system are at a concentration of from about 500 to about 800 mg wet weight/mL, or alternatively from about 500 to about 700 mg wet weight/mL, or alternatively from about 500 to about 600 mg wet weight/mL, or alternatively from about 600 to about 900 mg wet weight/mL, or alternatively from about 700 to about 900 mg wet weight/mL, or alternatively from about 800 to about 900 mg wet weight/mL, or alternatively from about 600 to about 800 mg wet weight/mL.

In another embodiment, the polymeric strands have an average molecular weight of from about 15,000 to about 2,000,000. In alternative embodiments, the polymeric strands have an average molecular weight of from about 15,000 to about 200,000, or alternatively from about 15,000 to about 20,000, or alternatively from about 150,000 to about 2,000,000, or alternatively from about 1,500,000 to about 2,000,000, or alternatively from about 100,000 to about 1,000,000, or alternatively from about 50,000 to about 1,500,000.

In another embodiment, the plurality of polymeric strands are obtained by a process comprising the steps of adding from about 0.01 to about 10 mol percent of a surfactant to a polymerization system comprising an effective amount of a monomer, or two or more different monomers, wherein the monomer or at least one of the two or more monomers comprise(s) one or more hydroxy and/or one or more ester groups, in an effective amount of a polar liquid or a mixture of polar liquids, wherein the polar liquid or at least one of the two or more polar liquids comprise(s) one or more hydroxy group. The monomer(s) are polymerized under suitable conditions to form a plurality of gel particles, each particle comprising a plurality of polymer strands. In a further aspect, the gel particles are isolated from the reaction composition. The particles formed by this method may be further processed or contain additional agents such as pharmaceutically active agents or biologicals, as described above. As is apparent to those of skill in the art, an effective amount of the additional agent is added to the polymerization solution.

In another embodiment, the liquids are selected from the group consisting of water, a (2C-7C)alcohol, a (3C-8C)polyol and a hydroxy-terminated polyethylene oxide. In a further embodiment, the liquids are selected from the group consisting of water, ethanol, isopropyl alcohol, benzyl alcohol, polyethylene glycol 200-600 and glycerine. In another embodiment, the liquid is water.

In another embodiment, the plurality of polymeric strands are obtained by a process comprising adding from about 0.01 to about 10 mol percent of an effective amount of a surfactant to a polymerization system comprising an effective amount of a monomer, or two or more different monomers, wherein the monomer or at least one of the two or more monomers comprise(s) one or more hydroxy and/or one or more ester groups, in an effective amount of a polar liquid or a mixture of polar liquids, wherein the polar liquid or at least one of the two or more polar liquids comprise(s) one or more hydroxy groups and a polymerizing the monomer(s) to form a plurality of gel particles, each particle comprising a plurality of polymer strands. In a further aspect, the process also comprises isolating the gel particles, wherein the process further comprises adding from about 0.1 to about 15% mol percent of a cross-linking agent to the polymerization system. In an alternate aspect, from about 0.5 to about 15%, or about 1 to about 10%, each in mol percent, of cross-linking agent are added to the system. The particles formed by this method may be further processed or contain additional agents such as pharmaceutically active agents or biologicals, as described above. As is apparent to those of skill in the art, an effective amount of the additional agent is added to the polymerization solution.

In another embodiment, the cross-linking agent is selected from the group consisting of ethylene glycol diacrylate, ethylene glycol dimethacrylate, 1,4-dihydroxybutane dimethacrylate, diethylene glycol dimethacrylate, propylene glycol dimethacrylate, diethylene glycol diacrylate, dipropylene glycol dimethacrylate, dipropylene glycol diacrylate, divinyl benzene, divinyltoluene, diallyl tartrate, diallyl malate, divinyl tartrate, triallyl melamine, N,N′-methylene bisacrylamide, diallyl maleate, divinyl ether, 1,3-diallyl 2-(2-hydroxyethyl) citrate, vinyl allyl citrate, allyl vinyl maleate, diallyl itaconate, di(2-hydroxyethyl) itaconate, divinyl sulfone, hexahydro-1,3,5-triallyltriazine, triallyl phosphite, diallyl benzenephosphonate, triallyl aconitate, divinyl citraconate, trimethylolpropane trimethacrylate and diallyl fumarate.

In another embodiment, the plurality of polymeric strands are obtained by a process comprising adding from about 0.01 to about 10 mol percent of a surfactant to a polymerization system comprising an effective amount of a monomer, or two or more different monomers, wherein the monomer or at least one of the two or more monomers comprise(s) one or more hydroxy and/or one or more ester groups, in an effective amount of a polar liquid or a mixture of polar liquids, wherein the polar liquid or at least one of the two or more polar liquids comprise(s) one or more hydroxy groups and polymerizing the monomer(s) to form a plurality of gel particles, each particle comprising a plurality of polymer strands and isolating the gel particles, wherein the process further comprises adding from about 0.1 to about 15% mol percent of a cross-linking agent to the polymerization system. In this aspect, the method further comprises adding an effective occluding amount of one or more pharmaceutically active agent(s) to the polar liquid(s) of the polymerization system prior to polymerization or after redispersing the gel particles in the liquid(s). The particles formed by this method may be further processed or contain additional agents such as pharmaceutically active agents or biologicals, as described above. As is apparent to those of skill in the art, an effective amount of the additional agent is added to the polymerization solution.

In another embodiment, the effective amount of the pharmaceutically active agent-containing gel particles occlude from about 0.1 to about 90 weight percent pharmaceutically active agent-containing liquid. In alternative embodiments, the effective amount of the pharmaceutically active agent-containing gel particles occlude from about 1 to about 90 weight percent pharmaceutically active agent-containing liquid, or alternatively from about 10 to about 90 weight percent, or alternatively from about 0.1 to about 70 weight percent, or alternatively from about 0.1 to about 50 weight percent, or alternatively from about 0.1 to about 20 weight percent, or alternatively from about 10 to about 50 weight per cent.

In another embodiment, the method comprises adding an effective amount of one or more first pharmaceutically active agent(s) to the polymerization system in an amount effective to give a first pharmaceutically active agent-containing liquid, wherein after polymerization, a portion of the first pharmaceutically active agent-containing liquid is occluded by the gel particles and isolating the gel particles containing the first pharmaceutically active agent(s) and then redispersing the gel particles in an effective amount of the polar liquid(s) and adding an effective amount of one or more second pharmaceutically active agent(s) to the suspension to give a second pharmaceutically active agent-containing liquid, wherein the first pharmaceutically active agent(s) may be the same as or different than the second pharmaceutically active agent(s) and the liquid of the first pharmaceutically active agent-containing liquid may be the same as or different than the liquid of the second pharmaceutically active agent-containing liquid.

In another embodiment, the pharmaceutical agent(s) further comprise(s) one or more pharmaceutically acceptable excipient(s). In another embodiment, the pharmaceutical agent(s) comprise(s) a peptide or protein.

Hydrogel Suspensions

This invention also provides a viscous, shape conforming gel, comprising a suspension of a plurality of gel particles as described above and exemplified below. In one aspect, this invention provides a viscous, shape conforming suspension of gel particles as described above, wherein the at least one monomer of the viscous, shape conforming gel is acrylic acid, methacrylic acid, 2-hydroxyethyl acrylate, 2-hydroxyethylmethacrylate, diethyleneglycol monoacrylate, diethyleneglycol monomethacrylate, 2-hydroxypropyl acrylate, 2-hydroxypropyl methacrylate, 3-hydroxypropyl acrylate, 3-hydroxypropyl methacrylate, dipropylene glycol monoacrylate, dipropylene glycol monomethacrylate, gylcidyl methacrylate, 2,3-dihydroxypropyl methacrylate, or glycidyl acrylate. In another embodiment, this invention provides a viscous, shape conforming gel, wherein the monomer(s) of the viscous, shape conforming gel is/are 2-hydroxyethyl methacrylate, 2-hydroxypropyl methacrylate, 3-hydroxypropyl methacrylate, glycerol methacrylate, or a combination thereof. In a further aspect, the polymer is comprised of one monomer only. In a further aspect, the polymer is a combination of two monomers of least one of which is e.g. 2-hydroxyethyl methacrylate, 2-hydroxypropyl methacrylate, 3-hydroxypropyl methacrylate or 2,3-dihydroxypropyl methacrylate. In a further aspect, the polymer is comprised of 2-hydroxyethyl methacrylate and 2,3-hydroxypropyl methacrylate monomers.

In another embodiment, this invention provides a viscous, shape conforming suspension of gel nanoparticles, the plurality of gel particles are about the same average diameter, are formed from one or more monomers and are of a narrow polydispersivity. In another embodiment, this invention provides a viscous, shape conforming suspension of gel nanoparticles wherein nanoparticles are of different average diameter and are formed from one or more monomers and are of a narrow polydispersivity. In another embodiment, the gel nanoparticles as described above are formed from one or more monomers and are of a broad polydispersivity.

In another embodiment, this invention provides a suspension of the nanoparticles as described above, wherein the plurality of gel particles of the viscous, shape conforming gel is at a concentration in the range of about 5-20% in the suspension system that results in cluster formation. Alternative concentrations within the scope of this invention include the range of about 5-10%, or alternatively about 5-15%, or alternatively about 10-20%, or alternatively about 15-20%, or alternatively about 10-15%, or alternatively about 6-19%, or alternatively about 7-18%, each of which results in cluster formation.

In another embodiment, this invention provides a viscous, shape conforming gel, wherein the surfactant of the viscous, shape conforming gel is at a concentration from about 0.005 weight percent to about 0.50 weight percent. In alternative embodiments, the effective amount of the surfactant is from about 0.005 weight percent to about 0.1 weight percent, or alternatively from about 0.005 weight percent to about 0.2 weight percent, or alternatively from about 0.005 weight percent to about 0.3 weight percent, or alternatively from about 0.005 weight percent to about 0.4 weight percent, or alternatively from about 0.05 weight percent to about 0.1 weight percent, or alternatively from about 0.05 weight percent to about 0.2 weight percent, or alternatively from about 0.05 weight percent to about 0.3 weight percent, or alternatively from about 0.05 weight percent to about 0.4 weight percent, or alternatively from about 0.05 weight percent to about 0.5 weight percent, or alternatively from about 0.006 weight percent to about 0.40 weight percent.

In another embodiment, this invention provides a viscous, shape conforming gel, wherein the average diameter of the gel particles of the viscous, shape conforming gel is from about 1 to about 1,000 nanometers. In alternative embodiments, the average diameter of the gel particles is from about 10 to about 1,000 nanometers, or, or alternatively from about 100 to about 1,000 nanometers, or alternatively from about 10 to about 100 nanometers, or alternatively from about 20 to about 1000 nanometers.

In another embodiment, this invention provides a viscous, shape conforming gel, wherein the average diameter of the gel nanoparticles of the viscous, shape conforming gel is from about 40 to about 800 nanometers. In alternative embodiments, the average diameter of the gel particles is from about 40 to about 500 nanometers, or alternatively from about 40 to about 300 nanometers, or alternatively from about 100 to about 800 nanometers, or alternatively from about 300 to about 800 nanometers, or alternatively from about 600 to about 800 nanometers, or alternatively from about 50 to about 700 nanometers.

In another embodiment, this invention provides a viscous, shape conforming gel, wherein the gel nanoparticles are at a concentration of from about 500 to about 900 mg wet weight/mL in the suspension system. In alternative embodiments, the gel particles in the suspension system are at a concentration of from about 500 to about 800 mg wet weight/mL, or alternatively from about 500 to about 700 mg wet weight/mL, or alternatively from about 500 to about 600 mg wet weight/mL, or alternatively from about 600 to about 900 mg wet weight/mL, or alternatively from about 700 to about 900 mg wet weight/mL, or alternatively from about 800 to about 900 mg wet weight/mL, or alternatively from about 600 to about 800 mg wet weight/mL. The amount of nanoparticles can be defined by dry weight and are as described above and incorporated herein by reference.

In another embodiment, this invention provides a viscous, shape conforming gel, wherein the polymer strands have an average molecular weight of from about 15,000 to about 2,000,000. In alternative embodiments, the polymeric strands have an average molecular weight of from about 15,000 to about 200,000, or alternatively from about 15,000 to about 20,000, or alternatively from about 150,000 to about 2,000,000, or alternatively from about 1,500,000 to about 2,000,000, or alternatively from about 100,000 to about 1,000,000, or alternatively from about 50,000 to about 1,500,000.

In another embodiment, this invention provides a viscous, shape conforming gel, wherein the plurality of polymeric strands is obtained by a process comprising:

i) adding from about 0.01 to about 10 mol percent of a surfactant to a polymerization system comprising a monomer, or two or more different monomers, wherein the monomer or at least one of the two or more monomers comprise(s) one or more hydroxy and/or one or more ester groups, in a polar liquid or a mixture of polar liquids, wherein the polar liquid or at least one of the two or more polar liquids comprise(s) one or more hydroxy groups;

ii) polymerizing the monomer(s) to form a plurality of gel particles, each particle comprising a plurality of polymer strands; and

iii) after polymerization, the liquid(s) are removed from the suspension such that the amount of liquid(s) remaining in the dry powder is less than 10% by weight when the percentage is based on the total weight of the dry powder.

The dry powder is then reconstituted to form the viscous gel as noted above. The viscoelastic gel is prepared by admixing between about 1 to about 50 percent by weight (dry), or alternatively between about 2 and 30% by weight (dry) or yet further between 8 and about 20% by weight (dry), in at least one polar liquid.

In another embodiment, this invention provides a viscous, shape conforming gel, wherein the liquids are selected from the group consisting of water, a (2C-7C)alcohol, a (3C-8C)polyol and a hydroxy-terminated polyethylene oxide.

In another embodiment, this invention provides a viscous, shape conforming gel, wherein the liquids are selected from the group consisting of water, ethanol, isopropyl alcohol, benzyl alcohol, polyethylene glycol 200-600 and glycerine.

In a further embodiment, this invention provides a viscous, shape conforming gel, wherein the liquid is water.

In another embodiment, this invention provides a viscous, shape conforming gel, wherein the gel further comprises from about 0.1 to about 15% mol percent of a cross-linking agent. In an alternate aspect, from about 0.5 to about 15%, or about 1 to about 10%, each in mol percent, of cross-linking agent are added to the system.

In another aspect, this invention provides a viscous, shape conforming gel, wherein the cross-linking agent is selected from the group consisting of ethylene glycol diacrylate, ethylene glycol dimethacrylate, 1,4-dihydroxybutane dimethacrylate, diethylene glycol dimethacrylate, propylene glycol dimethacrylate, diethylene glycol diacrylate, dipropylene glycol dimethacrylate, dipropylene glycol diacrylate, divinyl benzene, divinyltoluene, diallyl tartrate, diallyl malate, divinyl tartrate, triallyl melamine, N,N′-methylene bisacrylamide, diallyl maleate, divinyl ether, 1,3-diallyl 2-(2-hydroxyethyl) citrate, vinyl allyl citrate, allyl vinyl maleate, diallyl itaconate, di(2-hydroxyethyl) itaconate, divinyl sulfone, hexahydro-1,3,5-triallyltriazine, triallyl phosphite, diallyl benzenephosphonate, triallyl aconitate, divinyl citraconate, trimethylolpropane trimethacrylate and diallyl fumarate.

In another embodiment, this invention provides a viscous, shape conforming gel, wherein the gel further comprises one or more pharmaceutically active agents.

In another embodiment, this invention provides a viscous, shape conforming gel, wherein the pharmaceutically active agent containing gel particles occlude from about 0.1 to about 90 weight percent pharmaceutically active agent-containing liquid. In alternative embodiments, the effective amount of the pharmaceutically active agent-containing gel particles occlude from about 1 to about 90 weight percent pharmaceutically active agent-containing liquid, or alternatively from about 10 to about 90 weight percent, or alternatively from about 0.1 to about 70 weight percent, or alternatively from about 0.1 to about 50 weight percent, or alternatively from about 0.1 to about 20 weight percent, or alternatively from about 10 to about 50 weight percent.

In another embodiment, this invention provides a viscous, shape conforming gel, wherein the plurality of polymeric strands is obtained by a process comprising:

i) isolating the gel particles containing the first pharmaceutically active agent(s);

ii) redispersing the gel particles in an effective amount of the polar liquid(s); and

iii) adding one or more second pharmaceutically active agent(s) to the suspension to give a second pharmaceutically active agent-containing liquid, wherein the first pharmaceutically active agent(s) may be the same as or different than the second pharmaceutically active agent(s) and the liquid of the first pharmaceutically active agent-containing liquid may be the same as or different than the liquid of the second pharmaceutically active agent-containing liquid.

In another embodiment, this invention provides a viscous, shape conforming gel, wherein the pharmaceutically active agent(s) comprise one or more biomedical agent(s), which may be the same or different and are as defined above.

In another embodiment, this invention provides a viscous, shape conforming gel, wherein viscous, shape conforming gel as described above, wherein the pharmaceutical active agent(s) further comprise(s) one or more pharmaceutically acceptable excipient(s).

In another embodiment, this invention provides a viscous, shape conforming gel, wherein, the pharmaceutical active agent(s) comprise(s) a peptide or protein.

Medical Implant and Prosthesis

In one embodiment, this invention provides a medical prosthesis for tissue reconstruction comprising the viscous, shape conforming gel comprising a suspension of a plurality of gel particles as described herein in the medical prosthesis. In another embodiment, this invention provides a method for tissue reconstruction by implanting this medical prosthesis in a patient in need thereof. In one aspect, this invention provides a tissue reconstruction implant, comprising one or more of the viscous, shape conforming gel described above.

The following examples are intended to illustrate, not limit the invention.

Experimental

The viscous, shape conforming gels as described herein are formed by preparing a concentrated suspension of gel particles dispersed in a polar solvent(s) containing a surfactant to prevent self-aggregation.

The physical and chemical properties of the viscous, shape conforming gels can be manipulated such that they are stable and do not readily self-aggregate or degrade in the presence of the suspending liquid(s). Factors such as concentration and type of gel particles, size of the particles comprising the viscous gel and amount and type of surfactant present in the suspending medium will affect the resulting properties of the viscous gels. These gels can be produced to exhibit a variety of flow characteristics by changing concentration only. Properties such as hardness and elastic modulus can also be influenced by the composition of the gel particles present in the viscous gels. There is relationship between the maximum amount and type of gel particles that can be dispersed efficiently throughout the suspending liquid(s) and the amount of surfactant required to keep these particles, since they are in close proximity to each other as the concentration increases, from self-aggregating. For each proposed composition, this relationship can be empirically studied to optimize the performance and stability of these viscous, shape conforming gels for use as mammalian tissue reconstruction implants. If a catastrophic failure causing a rupture of the implant envelope occurs, the gel particles may leak into a physiological environment, and coalesce into a localized mass at the rupture point. Higher concentrations of surfactant, although desirable to keep the gel particles from self-aggregating, will prevent the particles from aggregating if exposed to a physiological environment. Thus, all of these factors must be considered when producing an optimized, stable, self-sealing viscous gel for use as a medical prosthesis. It is obvious to one skilled in the art that the amount and type of gel particles used the amount and type of surfactant used and the polar solvent(s) used to disperse the gel particles are important parameters in producing a variety of viscous gels that exhibit viscoelastic properties simulating various types of tissues in human body.

The gel particles are prepared in a polymerization system that consists of one or more monomers selected generally from those monomers that, on polymerization, provide a polymer that is capable of hydrogen bonding when in the presence of a polar liquid(s). General classes of monomers that have this capability include, without limitation, the hydroxy (2C-4C) alkyl methacrylates and the hydroxy (2C-4C) alkyl acrylates such as 2-hydroxyethylmethacrylate and acrylate; the dihydroxy (2C-4C) alkyl 2-alkenoates such as 2,3-dihydroxypropylmethacrylate; the hydroxy ((2C-4C) alkoxy (2C-4C) alkyl) alkenoates such as 2-hydroxyethoxyethyl acrylate and methacrylate; the (1C-4C) alkoxy (1C-4C) alkyl methacrylates, e.g., ethoxyethyl methacrylate; the 2-alkenoic acids, such as acrylic and methacrylic acid; the (1C-4C) alkoxy (2C-4C) alkoxy (2C-4C) alkyl) alkenoates such as ethoxyethoxyethyl acrylate and methacrylate; the N,N-di(1C-4C) alkylaminoalkyl-2-alkenoates such as diethylaminoethylacrylate and methacrylate and the vicinyl epoxy (1C-4C) alkyl 2-alkenoates such as glycidyl methacrylate and combinations thereof.

Specific examples of monomers include 2-hydroxyethyl acrylate, 2-hydroxyethyl methacrylate, diethylene glycol monoacrylate, diethylene glycol monomethacrylate, 2-hydropropyl acrylate, 2-hydroxypropyl methacrylate, 3-hydroxypropyl acrylate, 3-hydroxypropyl methacrylate, dipropylene glycol monomethacrylate, dipropylene glycol monoacrylate, glycidyl methacrylate, 2,3-dihydroxypropyl methacrylate, N,N-dimethlaminoethyl methacrylate N,N-dimethylaminoethyl acrylate, and mixtures thereof. One specific monomer is 2-hydroxyethyl methacrylate (HEMA) or 2,3-hydroxypropyl methacrylate which may be the sole monomer type or it may be at least one of the monomer types.

Co-monomers that are not capable of hydrogen bonding may be added to the polymerization system to modify the physical and chemical characteristics of the resulting gel particles. Examples of co-monomers that may be used in conjunction with the above monomers are, without limitation, acrylamide, N-methylmethacrylamide, N,N-dimethacrylamide, methylvinylpyrrolidone,

A cross-linking agent also may be added to the polymerization system to strengthen the three-dimensional structure of the resulting gel particles. The cross-linking agent can be non-degradable, such as, without limitation, ethylene glycol diacrylate or dimethacrylate, 1,4-butylene dimethacrylate, diethylene glycol dimethacrylate, propylene glycol dimethacrylate, diethylene glycol dimethacrylate, dipropylene glycol dimethacrylate, diethylene glycol diacrylate, dipropylene glycol diacrylate, divinyl benzene, divinyltoluene, triallyl melamine, N,N′-methylene bisacrylamide, diallyl maleate, divinyl ether, diallyl monoethylene glycol citrate, vinyl allyl citrate, allyl vinyl maleate, divinyl sulfone, hexahydro-1,3,5-triallyltriazine, triallyl phosphite, diallyl benzene phosphonate, a polyester of maleic anhydride with triethylene glycol, diallyl aconitrate, divinyl citraconate, trimethylolpropane trimethacrylate and diallyl fumarate. Other non-degradable cross-linking agents will become apparent to those skilled in the art based on the disclosures herein and are within the scope of this invention.

A particular liquid for use in both the polymerization system and the suspension system of this invention is water, in which case, the particles are hydrogel particles.

Certain organic liquids may also be used in the methods of this invention. In general, they should have boiling points above about 60° C., or alternatively above about 80° C., 100° C., 120° C., 140° C. 160° C., 180° C. or about 200° C. The use of these liquids results in the polymerization of gel particles and the production of viscous, shape conforming gels. Organic liquids that are particularly useful in forming the viscous gels of this invention are water-miscible oxyalkylene polymers, e.g., the polyalkylene glycols, especially those characterized by a plurality of oxyethylene (—OCH₂CH₂—) units in the molecule and a boiling point above about 200° C.

Particular organic liquids that may be used in the methods of this invention are biologically inert, non-toxic, polar, water-miscible organic liquids such as, without limitation, ethylene glycol, propylene glycol, dipropylene glycol, butanediol-1,3, butanediol-1,4, hexanediol-2,5,2-methyl-2,4-pentanediol, heptanediol-2,4,2-ethyl-1,3-hexanediol, diethylene glycol, triethylene glycol, tetraethylene glycols, and the higher polyethylene glycols and other water-soluble oxyalkylene homopolymers and copolymers having a molecular weight up to about 2000, preferably up to about 1600. For example, without limitation, hydroxy-terminated polymers of ethylene oxide having average molecular weights of 200-1000, water-soluble oxyethyleneoxypropylene polyol (especially glycol) polymers having molecular weights up to about 1500, preferably up to about 1000, propylene glycol monoethyl ether, monoacetin, glycerine, tri(hydroxyethyl) citrate, ethylene glycol monomethyl ether, ethylene glycol monoethyl ether, di(hydroxypropyl) oxalate, hydroxypropyl acetate, glyceryl triacetate, glyceryl tributyrate, liquid sorbitol ethylene oxide adducts, liquid glycerine ethylene oxide adducts, diethylene glycol monomethyl ether, diethylene glycol monoethyl ether, and ethylene glycol diacetate, may be used.

In an embodiment of this invention, hydrogel particles, having nominal sizes in the 10⁻⁹ meters to the 10⁻⁶ m range are produced by redox, free radical or photo-initiated polymerization in water containing a surfactant. In this manner, particles of relatively narrow polydispersivity can be produced. However, in certain drug delivery applications, it may be desireable to produce particles of a broad polydispersivity or use two or more groups of particles of different size but narrow polydispersivity within each size to comprise the viscoelastic gel contained within a medically acceptable envelope of an implant. If an inadvertent rupture occured, an aggregate would form at the rupture site, and a biologically active substance would be released systemically or locally over a prolonged period of time. The release rate, to some extent, can be regulated based on the composition, size and polydispersivity of the particles comprising the viscoelatic gel. It is obvious to one skilled in the art, that a biologically active substance or substances can be added to the suspending medium comprising the viscoelastic gel and/or added during the polymerization step to produce gel particles that occlude the active. Thus, the versatility of the technology allows for a variety of drug delivery applications, including, without limitation, the release of actives at the implant rupture site and the release of actives from the viscoelastic gel particles and/or suspending medium through the implant shell. The dual release of actives alone, or in combination, can also be accomplished using different sizes and polydispersivities of nanoparticles comprising the viscoelastic gel. and

Prior to redispersing the gel particles into a polar liquid(s), it may be desirable to treat the suspension system to remove unreacted monomer(s), surfactant and non-occluded biologically active substance from the liquid of the suspension system and/or to remove unreacted monomer(s) and surfactant from the water absorbed by the particles. Techniques such as, without limitation, dialysis, extraction or tangential flow filtration may be used to clean up the particles and the suspension system. It may also be desirable to exchange the surfactant used during the polymerization and formation of gel particles for a more pharmaceutically acceptable one. The purified gel particles, with or without an occluded biologically active substance, are then isolated by techniques such as, without limitation, spray drying or lyophilization and the dried particles are resuspended at a high concentration in the polar liquid(s) containing a surfactant and with or without another biologically active substance. The concentration of gel particles in the viscous, shape conforming gels may be, as described herein, for example, in the range from about 300 to about 1200 mg wet weight/mL, more preferably from about 500 to 900 mg weight weight/mL. The amount of surfactant present in the liquid(s) is in the range of about 0.005 to about 0.50 weight percent, in another aspect from about 0.01 to 0.10 weight percent.

The viscous, shape conforming gels contained within a medically, acceptable implantable envelope material will generally be prepared so as to be stable under selected storage conditions. However, if they are subjected to physiological conditions of ionicity, pH and the like, such as in the case of an inadvertent rupture, the gel particles will undergo a reduction in zeta potential and subsequent coalescence and localized aggregation will occur at the rupture site. This is an added safety feature, namely a “self-sealing” aspect not found with any commercially available implants. For example, in the case of silicone implants, the fluid inside the implant is deemed “unsafe”. Thus if a rupture occurs, bodily tissues, both locally and systemically become exposed to this toxic substance. With the viscous gel implants of the present invention, no toxic materials comprise the implant. If an inadvertent rupture occurs, the surrounding tissue becomes exposed only to a biologically safe material, and since the aggregate remains together as a solid mass, there are no systemic toxicity concerns. Also, if necessary, the aggregate can be surgically removed. An additional attribute of the viscous gels of the present invention is the ability to include one or more biologically active substances occluded within the individual gel particles and/or throughout the suspending polar liquid(s). The viscous gel materials could provide, if desired, a controlled delivery of these active agents through a drug permeable envelope material into the surrounding tissue area. This is particularly advantageous for treating localized infection using an antibiotic, antimicrobial or other compound as a result of implantation surgery and the possibility of delivering an anti-rejection pharmaceutical agent.

Numerous factors will affect the chemical and physical characteristics of the viscous, shape conforming gels of this invention. One is the molecular weight of the polymer used to form the individual hydrogel particles. It has been found that hydrogel particles consisting of low molecular weight polymers will generally not form viscous gels as compared to higher molecular weight polymers at the same concentration, and these particles will not aggregate when exposed to a physiological environment. Thus, higher molecular weight polymers are used in this invention. While the use of cross-linking agents can ameliorate some of the problems associated with low molecular weight polymers, too much cross-linking agent may be detrimental. If the hydrogel particles contain a large amount of cross-linking agent and/or if the cross-linking agent is highly hydrophobic, the resulting polymeric network may not permit optimal absorption of liquid resulting in less desirable viscous gels, so the polymers that comprise the gel particles of this invention have molecular weights in the range of about 15,000 to about 2,000,000 Da or alternatively from about 20,000 to about 1,500,000 Da, or alternatively from about 25,000 to about 1,000,000 Da. This may be accomplished by selecting an appropriate commercial monomer, by using a polymerization system that gives polymers of in the desired molecular weight range or by including a cross-linker in the polymerization system to join together short polymer strands to reach the desired molecular weight range.

Particle size will also affect the characteristics of the viscous gels. It has been determined that smaller gel particles will generally absorb liquid more easily and will give preferred viscous, shape conforming gels suitable as a mammalian tissue reconstruction implant. Gel particles having sizes, again as characterized by their average diameters, in the range of about 1 to about 1,000 nm, or alternatively from about 10 to about 800 nm, or alternatively between about 50 to about 600 nm, can be used.

If a cross-linking agent is used, its chemical composition and the amount used, i.e., the resulting cross-linking density, will affect the characteristics of the particles as previously discussed and thereupon will affect the characteristics of the viscous gels formed. The amount of cross-linking agent used in preparing gel particles of this invention is in the range of about 0.001 to about 10, or alternatively about 0.1 to about 2 mol percent of monomer.

The molecular weight and chemical composition of the suspension liquids and the amount and type of surfactant used will also affect the resulting viscous, shape conforming gels since a large amount of liquid is absorbed by the particles which is a function of how much these gel particles swell in the respective polar liquid(s) which affects flowability. The swelling occurs to a larger extent in lower molecular weight, polar liquids as compared to a reduced swelling in similar higher molecular weight liquids. For instance, as noted previously, water can be used both for the polymerization system and the suspension system. Deoxycholate in the viscous, shape conforming gels is a specific surfactant that may be used in the materials and methods described herein. This medically safe surfactant keeps the swollen gel particles stabilized at high concentrations to allow viscous gels to be produced without self-aggregating. Other pharmaceutically acceptable surfactants can be used in these viscous gel suspensions and a variety of surfactants are also suitable for use in polymerizing monomers to produce gel particles that comprise the viscous, shape conforming gels of this invention.

The concentration of gel particles in the suspension system will affect the characteristics of the resulting viscous, shape conforming gels primarily due to the fact that at higher concentrations, the flow characteristics are reduced and the viscosity increases substantially since the particles tend to coalesce into particle clusters and the dispersion approaches that of a viscoelastic material without self-aggregating into a solid mass.

It is also apparent to one skilled in the art, that there is an appropriate amount of surfactant required to keep a specific concentration of gel particles suspended in theses viscous, shape conforming gels to prevent self-aggregation. The chemical composition and amount of surfactant used will affect the physical and chemical characteristics of these viscous, shape conforming gels of this invention. As noted above, the amount of surfactant present in the liquid(s) is in the range of about 0.01 to about 0.10 weight percent. These concentrations are variable depending upon the specific surfactant used and the type and amount of gel particles and polar solvent(s) used to produce these viscous gels.

The various parameters discussed above are, of course, inter-dependent. For example, without limitation, the physical characteristics of these viscous gels are directly proportional to the concentration, type and particle size of gel particles used in suspension at a given concentration and type of surfactant and polar liquid(s) used. Smaller, gel particles suspended in water in the presence of a surfactant at a higher concentration produce a more viscoelastic gel than utilizing a lower concentration of gel particles. Suspended larger gel particles will provide viscous gels, but have a higher probability of self-aggregating into a solid mass. Also, a viscous shape conforming gel, comprising gel particles composed of poly-2-hydroxyethyl methacrylate, will behave differently than a gel composed of poly-2-hydroxypropyl methacrylate. At the same concentration of gel particles, and using the same concentration and type of surfactant and polar liquid, poly-HEMA gels will be softer than those composed of poly-HPMA. Mixtures of both types of polymer gel particles will provide properties somewhere in between. Also, gel particles of copolymers comprised of HEMA and HPMA will also behave differently. This is another major attribute of this invention, that is, the ability to tune the “viscoelasticity” and offer a variety of viscous, shape conforming gels that can be used to simulate different types of mammalian tissue. To the best of Applicants' knowledge, no other commercially available implant can provide such a selection.

In one embodiment of this invention, hydrogel particles are produced by polymerizing non-ionic monomers in water containing a surfactant. The suspension of hydrogel particles is treated to remove unreacted monomer and other impurities. The suspension of gel particles is spray dried or lyophilized to isolate the particles, and the dry, gel particles are resuspended in water at a concentration approaching 1000 mg/mL wet weight in water containing deoxycholate.

This viscous gel is then transferred into a medically acceptable, implantable envelope material of a specific size and shape used in preparing a medical prosthesis for mammalian tissue reconstruction.

In an embodiment of this invention, a biologically active agent is dissolved or resuspended in an aqueous suspension of a high concentration of hydrated hydrogel particles, and the viscous gel in placed in a medically accepted, semi-permeable shell material for use as a medicated mammalian tissue reconstruction implant. After implantation, the biologically active agent will migrate out of the implant, at a controlled rate, through the drug permeable envelope into the surrounding tissue to treat, for example, infection and biological rejection of this device.

Another embodiment of this invention involves dissolving or suspending the biologically active agent in the polymerization system prior to polymerization. As the polymerization reaction proceeds and hydrogel particles form, liquid containing the biologically active substance is occluded by the forming particles. Non-occluded biologically active agent is then removed when the particles are treated to remove excess monomer and surfactant. The suspension of biologically active substance-containing particles is then isolated, dried and the gel particles containing an occluded biologically active agent are resuspended at a high concentration in water containing a surfactant to produce a viscous, medicated gel implant.

A combination of the above approaches is an embodiment of this invention. One biologically active agent can be occluded within the gel particles during polymerization and another or the same biologically active substance can be present in the suspending medium when the dry, gel particles with occluded drug are resuspended at a high concentration to produce the medicated gel implant. This approach would be most viable if it is desirable to mitigate the burst release of an active in order to obtain a near “zero order release” of an active, or to release two different actives for the treatment of the same or different indications.

The type and amount of an agent that can be occluded by a gel particle of this invention depends upon a variety of factors. First and foremost, the agent cannot interfere, due to its size, surface charges, polarity, steric interactions, etc., with the formation of discrete gel particles. Once it is determined that the foregoing is not a problem, the size of the hydrogel particles most directly affects the quantity of substance that can be incorporated. The size of the particles themselves will dictate the maximum amount of agent that can be occluded. Relatively small agents, such as individual antibiotic molecules, may be entrapped in small gel particles while it will be much more difficult to occlude substantially larger agents such as monoclonal antibodies, proteins, peptides and other macromolecules. With these larger compounds, it may be desirable to introduce them in the suspending medium when the viscous, shape conforming gels are produced by redispersing the gel particles at a high concentration.

Using the methods herein, precise control of delivery kinetics can be achieved. That is, gel particles of differing sizes and chemical compositions can be loaded with a particular agent and, depending on the characteristics of the various particles, the agent can be released over virtually any desired timeframe. In addition, some of the substance might be occluded in the gel particles and some might be present in the suspending medium between the particles comprising the viscous gel to provide even more delivery flexibility.

Thus, the present invention provides an extremely versatile, biocompatible implant material with a potential drug delivery platform, in particular with regard to biologically active agent delivery and most particularly with regard to pharmaceutical agent delivery. The ability to provide a biologically safe, viscous gel material for mammalian tissue reconstruction is unique in every respect as compared to the present state of the art mammalian tissue reconstruction implant materials. An additional benefit is the self-sealing aspect of these viscous gels, such that if an inadvertent rupture occurs, only a localized formation of a solid aggregate mass results instead of leakage of toxic fluid to the surrounding tissue. If necessary, this biologically safe material can then be surgically removed. These attributes of the viscous, shape conforming gels of the present invention are novel and will provide a new class of mammalian tissue reconstruction implants to address all of the problems associated with current implant technology.

These and may other uses for these viscous, shape conforming gels of this invention will become apparent to those skilled in the art based on the disclosures herein. Such uses are within the scope of this invention.

It will be appreciated by one of skill in the art that the embodiments summarized above may be used together in any suitable combination to generate additional embodiments not expressly recited above, and that such embodiments are considered to be part of the present invention.

### EXAMPLES

1. Hydrogel Nanoparticle Synthesis

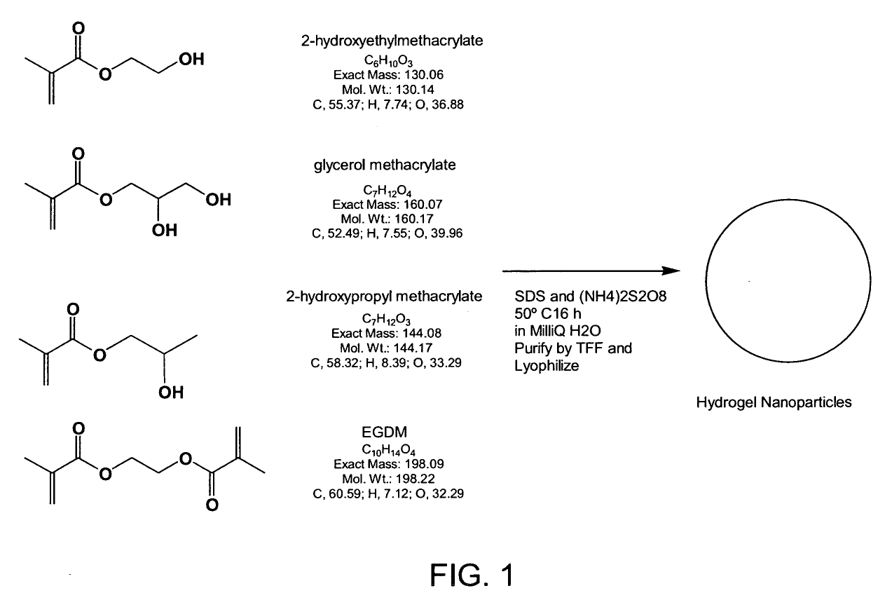

Hydrogel nanoparticles are synthesized in a free radical polymerization from 2-hydroxyethylmethacrylate, 2-hydroxypropylmethacrylate, or glycerolmethacrylate. The general scheme for the synthesis of these materials is shown in FIG. 1.

A general synthetic procedure for the formation of a laboratory scale batch of nanoparticles follows:

1). Synthesis of poly(2-hydroxyethylmethacrylate) nanoparticles (pHEMA nps)
a). Into a 2 liter media bottle, weigh ingredients.
b). Cover the bottle with foil and immerse in a 50° C. thermostated water bath overnight (ca. 16 h).
c). Remove the media bottle from the water bath and cool to ambient temperature.
d). Determine the nanoparticle wet-weight by removing two×3 mL aliquots from the nanoparticle dispersions and ultra-centrifuging these samples for 1 h at 70 k rpm. Decant off the supernatant and weigh the as-formed nanoparticle aggregate and determine the wet weight per unit volume (mg/mL dispersion). This provides an estimate for the nanoparticle yield.
e). Remove several drops of the dispersion and determine the nanoparticle sizes, size range, polydispersivity and Zeta Potential (surface charge) using the Malvern NanoZS instrument for experimental data analysis.
f). Purify the nanoparticle dispersion by TFF (removes residual monomers, salts and SDS, while replacing the SDS using a TFF makeup feed of 0.01 wt. % sodium deoxycholate (DOC) solution. 1 g DOC to 10 liters of MilliQ water). This process maintains the Zeta Potential (ZP) at a suitable level; i.e., −35 mV≧ZP nps≧−25 mV, stabilizing the nanoparticles as a dispersion preventing unwanted nanoclustering and nanoparticle aggregation. Pump the nanoparticle dispersion through 1,000,000 molecular weight cutoff filters and collect seven×2 liter volumes of permeate while maintaining the nanoparticle dispersion reservoir at 2 liters with the 0.005 wt % DOC TFF makeup feed in a continuous flow system.
g). Freeze the dispersion in a liquid nitrogen bath and lyophilize the material.
h). Isolate the lyophilized powder and transfer it into a tarred plastic bottle for storage

The particle size for nanoparticles changes during lyophilization. Lyophilized nanoparticles can be redispersed in water or a suitable polar solvent

Table 1. below shows changes in particle size before and after lyophilization for nanoparticles for different hydrogel polymers and copolymers synthesized at 40 mg/mL in water wet weight (approximately 10 mg/mL dry polymer weight) and redispersed at the same concentration:

TABLE 1

| | Size after | Size after |
|---|---|---|
| Sample | Synthesis | Lyophilization |
| pHEMA | 38 nm | 154 nm |
| pHPMA | 42 nm | 186 nm |
| 50:50 pHEMA:HPMA | 56 nm | 248 nm |
| 85:15 pHEMA:HPMA | 42 nm | 168 nm |
| 33:33:33 | 56 nm | 131 nm |
| pHEMA:HPMA:GMA | | |

The following specific examples illustrate the synthesis of several hydrogel nanoparticles.

2. Preparation of Cross-Linked poly-2-hydroxypropyl methacrylate (pHPMA) Nanoparticles.

A 150 mL media bottle equipped with a stir bar was charged with 2.532 g (17.5 mmol) of hydroxypropylmethacrylate (HPMA) monomer, 52.73 mg (0.266 mmol) of ethylene glycol dimethacrylate (EGDM) crosslinker, 107.6 mg (0.3730 mmol) sodium dodecylsulfate (SDS), and 118 mL of nitrogen degassed Milli-Q H₂O. The bottle was closed and stirred to form a clear solution. In a separate vial, 83 mg of K₂S₂O₈ was dissolved into 2 mL of Milli-Q H₂O and added to the media bottle while stirring. The media bottle with clear solution was transferred into a 40° C. water bath and held at constant temperature for 12 hours. The resulting suspension of hydrogel nanoparticles had an opalescent blue color. The particles were analyzed by laser light scattering and found to have an average particle size of 21.3 nm and a size range from 14 nm to 41 nm. The suspension had approximately 1% solid polymer by mass. To date, this suspension of hydrogel nanoparticles resisted flocculation or aggregation for two years at room temperature. The suspension is then further processed as described above.

3. Preparation of Cross-Linked Nanoparticles Composed of Copolymers of HPMA and methacrylic acid (MAA), poly (HPMA-co-MAA).

Using the synthetic method of described in paragraph 3, hydrogel nanoparticles were produced using HPMA monomer and methacrylic acid. Table 2 shows the relative masses and mmol of monomers added to the 150 mL media bottles.

TABLE 2

| | Mass | Mmol | | |
|---|---|---|---|---|
| Sample | HPMA | HPMA | Mass MAA | mmol MAA |
| 95:5 | 2.40 g | 16.63 | 75.32 mg | 0.875 |
| pHPMA:MAA | | | | |
| 90:10 | 2.27 g | 15.75 | 150.65 mg | 1.75 |
| pHPMA:MAA | | | | |
| 80:20 | 2.02 g | 14.01 | 301.32 mg | 3.5 |
| pHPMA:MAA | | | | |
| 70:30 | 1.77 g | 12.25 | 443.98 mg | 5.25 |
| pHPMA:MAA | | | | |

Each media bottle was then charged with 52.73 mg (0.266 mmol) EGDM, 107.6 mg (0.3730 mmol) sodium dodecylsulfate (SDS), and 118 mL of nitrogen degassed Milli-Q H₂O. The bottles were capped and stirred for 30 minutes at room temperature. In a separate vial, 83 mg of K₂S₂O₈ was dissolved into 2 mL of Milli-Q H₂O and added to the media bottle while stirring. The media bottle with clear solution was transferred into a 40° C. water bath and held at constant temperature for 12 hours. The resulting suspension of hydrogel nanoparticles had an opalescent blue color. The particles were analyzed by laser light scattering and Table 3 shows the average size and particle size range.

TABLE 3

| | Average | Size range |
|---|---|---|
| Sample | size (nm) | (nm) |
| 95:5 | 24.3 | 17-35 |
| pHPMA:MAA | | |
| 90:10 | 27.1 | 20-35 |
| pHPMA:MAA | | |
| 80:20 | 24.0 | 20-30 |
| pHPMA:MAA | | |
| 70:30 | 31.8 | 20-60 |
| pHPMA:MAA | | |

4. Preparation of Cross-Linked poly-glycerol methacrylate (pGMA) Nanoparticles.

A 2000 mL media bottle equipped with a stir bar was charged with 53.6 g (335.05 mmol) of glycerolmethacrylate (GMA) monomer, 80 mg (0.404 mmol) of EGDM crosslinker, 20.4 g (7.09 mmol) sodium dodecylsulfate (SDS), and 2000 mL of nitrogen-degassed Milli-Q H₂O. The bottle was closed and stirred to form a clear solution. In a separate vial, 1.44 g (6.31 mmol) of (NH₄)₂S₂O₈ was dissolved into 20 mL of Milli-Q H₂O and added to the media bottle while stirring. The media bottle with clear solution was transferred into a 50° C. water bath and held at constant temperature for 12 hours. The resulting suspension of hydrogel nanoparticles had an opalescent blue color. The particles were analyzed by laser light scattering and found to have an average particle size of 156.5 nm and a nominal peak width of 49.37 nm. The suspension had approximately 2% solid polymer by mass. To date, this suspension of hydrogel nanoparticles resisted flocculation or aggregation for 1.5 years at room temperature. After ultracentrifugation, the resulting aggregate contained 84.5% water. The powder is then further processed as described above.

5. Preparation of Cross-Linked Nanoparticles Composed of Copolymers of HEMA and GMA, poly (HEMA-co-GMA).

Using the synthetic method of Paragraph 6, nanoparticles were produced using HEMA and glycerol methacrylate monomers. Table 4 shows the relative masses and mmol of monomers added to the 2000 mL media bottles.

TABLE 4

| | Mass | | mmol | | mmol |
|---|---|---|---|---|---|
| Sample | HEMA | | HEMA | Mass GMA | GMA |
| 90:10 | 40.0 | g | 307.36 | 4.47 g | 27.78 |
| pHEMA:GMA | | | | | |
| 75:25 | 33.35 | | 256.30 | 11.11 g | 69.46 |
| pHEMA:GMA | | | | | |

Each media bottle was then charged with 80 mg (0.404 mmol) of EGDM crosslinker, 20.4 g (7.09 mmol) sodium dodecylsulfate (SDS), and 2000 mL of nitrogen-degassed Milli-Q H₂O. The bottles were closed and stirred to form clear solutions. In two separate vials, 1.44 g (6.31 mmol) of (NH₄)₂S₂O₈ was dissolved into 20 mL of Milli-Q H₂O and added to the 2000 mL media bottles while stirring. The media bottles with clear solution were transferred into a 50° C. water bath and held at constant temperature for 12 hours. The resulting suspensions of hydrogel nanoparticles were opalescent blue in color. The particles were analyzed by laser light scattering and Table 5 shows the average size and particle size range.

TABLE 5

| | Average | Peak width |
|---|---|---|
| Sample | size (nm) | (nm) |
| 90:10 | 160.3 nm | 46.56 nm |
| pHEMA:GMA | | |
| 75:25 | 49.37 nm | 40.87 nm |
| pHEMA:GMA | | |

To date, this suspensions of poly-co-HPMA:GMA nanoparticles resisted flocculation or aggregation for over 6 months at room temperature. In addition, the suspensions formed elastic shape retentive aggregates when subjected to ultracentrifugation. The suspension is then further processed as described herein.

6. Preparation of Cross-Linked poly(methacrylic acid) (pMAA) Nanoparticles.

A 150 mL media bottle equipped with a stir bar was charged with 1.505 g (17.5 mmol) of methacrylic acid (MAA) monomer, 52.73 mg (0.266 mmol) of ethylene glycol dimethacrylate(EGDM) crosslinker, 107.6 mg (0.3730 mmol) sodium dodecylsulfate (SDS), and 118 mL of nitrogen degassed Milli-Q H₂O. The bottle was closed and stirred to form a clear solution. In a separate vial, 83 mg of K₂S₂O₈ was dissolved into 2 mL of Milli-Q H₂O and added to the media bottle while stirring. The media bottle with clear solution was transferred into a 40° C. water bath and held at constant temperature for 12 hours. The resulting suspension of hydrogel nanoparticles had an opalescent blue color. The particles were analyzed by laser light scattering and found to have an average particle size of 21.3 nm and a size range from 14 nm to 41 nm. The suspension had approximately 1% solid polymer by mass. To date, this suspension of hydrogel nanoparticles resisted flocculation or aggregation for two years at room temperature. Also, a solid, shape retentive plug resulted after ultracentrifuging twenty milliliters of a 0.4% (w/w) suspension of poly-methacrylic acid nanoparticles at 100,000 rpm. The suspension is then further processed as described herein.

7. Preparation of poly(2-methoxyethyl methacrylate) (pMEMA) Nanoparticles.

A 250 mL media bottle equipped with a stir bar was charged with 4.2g of 2-methoxyethyl methacrylate (MEMA) monomer, 300 mg sodium dodecylsulfate (SDS), and 200 mL Milli-Q H₂O. The bottle was closed and stirred to form a clear solution. In a separate vial, 141 mg of K₂S₂O₈ was dissolved into 5 mL of Milli-Q H₂O and added to the media bottle while stirring. The media bottle with clear solution was transferred into a 50° C. water bath and held at constant temperature for 16 hours. The resulting suspension of hydrogel nanoparticles had an opalescent blue color. The particles were analyzed by laser light scattering and found to have an average particle size of 52.4 nm and a size range from 12 nm to 103 nm. The suspension had approximately 2.1% solid polymer by mass. To date, this suspension of hydrogel nanoparticles resisted flocculation or aggregation at room temperature. Also, a solid, shape retentive plug resulted after ultracentrifuging 5 milliliters of a 2.1 (w/w) suspension of poly(2-methoxyethyl methacrylate) nanoparticles at 100,000 rpm. The suspension is then further processed as described herein.

8. Preparation of poly(glycidyl methacrylate) (pGCMA) Nanoparticles.

A 250 mL media bottle equipped with a stir bar was charged with 4.2 g of glycidyl methacrylate (GCMA) monomer, 300 mg sodium dodecylsulfate (SDS), and 200 mL Milli-Q H₂O. The bottle was closed and stirred to form a clear solution. In a separate vial, 141 mg of K₂S₂O₈ was dissolved into 5 mL of Milli-Q H₂O and added to the media bottle while stirring. The media bottle with clear solution was transferred into a 50° C. water bath and held at constant temperature for 16 hours. The resulting suspension of hydrogel nanoparticles had an opalescent blue color. The particles were analyzed by laser light scattering and found to have an average particle size of 65.2 nm and a size range from 17 nm to 101 nm. The suspension had approximately 2.1% solid polymer by mass. To date, this suspension of hydrogel nanoparticles resisted flocculation or aggregation at room temperature. Also, a solid, shape retentive plug resulted after ultracentrifuging 5 milliliters of a 2.1 (w/w) suspension of poly(glycidyl methacrylate) nanoparticles at 100,000 rpm. The suspension is then further processed as described herein.

9. Attempt to Produce poly(2-sulfoethyl methacrylate) (pSEMA) Nanoparticles.

A 250 mL media bottle equipped with a stir bar was charged with 4.2 g of 2-sulfoethyl methacrylate (SEMA) monomer, 300 mg sodium dodecylsulfate (SDS), and 200 mL Milli-Q H₂O. The bottle was closed and stirred to form a clear solution. In a separate vial, 141 mg of K₂S₂O₈ was dissolved into 5 mL of Milli-Q H₂O and added to the media bottle while stirring. The media bottle with clear solution was transferred into a 50° C. water bath and held at constant temperature for 16 hours. The resulting mixture did not produce the characteristic opalescent blue color of other suspensions. Laser light scattering indicated little to no particles capable of scattering light at the described wavelength. The suspension had approximately 2.1% solid polymer by mass upon precipitation in sodium chloride solution. No centrifugation was performed.

10. Formation of Viscous Shape Conforming Gels

Viscous shape conforming gels are formed by dispersing the dehydrated hydrogel nanoparticle powder in water. A typical gel formation is described below:

1). Formation of Viscous Shape Conforming Gel
a. Disperse 100 mg of lyophilized pHEMA nanoparticle powder in 2 mL of 0.02 wt % deoxycholate in water.
b. Allow suspension to stand at room temperature for approximately 8 hours.

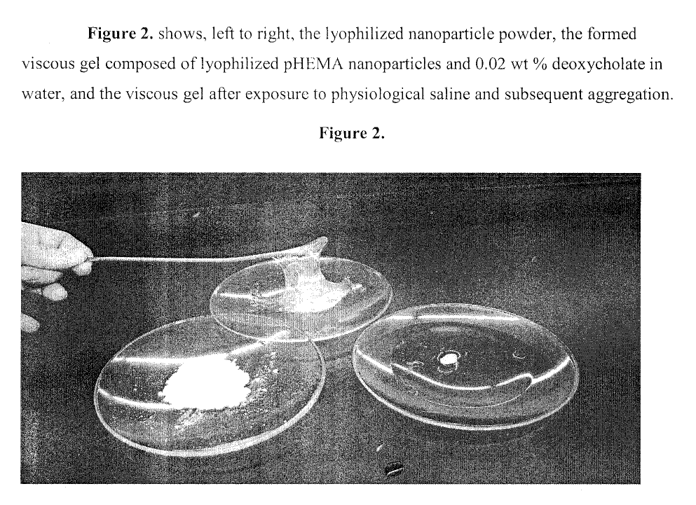

FIG. 2 shows the image of a nanoparticle powder, a formed shape conforming gel and a gel that has been exposed to physiological saline to form a shape retentive aggregate.

11. Physical Properties of Viscous Shape Conforming Gels

The chemical composition of nanoparticles in lyophilized hydrogel nanoparticle powder can affect the physical properties of viscous shape conforming gels.

Table 6. shows the relative viscosities for gels composed of different types of nanoparticles, including homopolymers, copolymers, and mixtures of homopolymers at 50 mg/mL(dry polymer weight) in 0.02 wt % deoxycholate in water.

TABLE 6

| Sample | Viscosity (cps) |
|---|---|
| pHEMA | 6.8 |
| pHPMA | 13.4 |
| 50:50 pHEMA:HPMA | 8.2 |
| 85:15 pHEMA:HPMA | 8.6 |
| 33:33:33 | 4.1 |
| pHEMA:HPMA:GMA | |
| 90:10 pHEMA/pHPMA | 7.2 |
| 85:15 pHEMA/pHPMA | 8.6 |
| 75:25 pHEMA/pHPMA | 8.8 |
| 50:50 pHEMA/pHPMA | 9.1 |

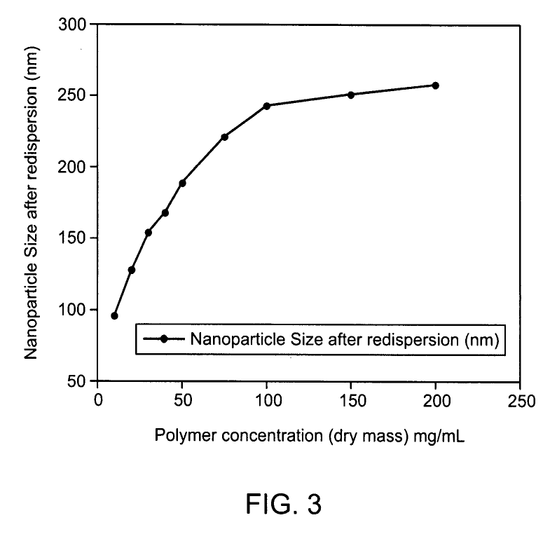

Nanoparticles increase in size in a viscous shape conforming gel as the concentration of particles increases. FIG. 3 shows the change in nanoparticle size as the concentration of particles in a gel increases from 10 mg/mL in water to 200 mg/mL (dry weight), indicating cluster formation.

As shown in FIG. 3, the size of the nanoparticles increases in the gel as the concentration is increased. The nanoparticle size increases from the initial 40-50 nm to approximately 250 nm at a concentration of 200 mg/mL in water.

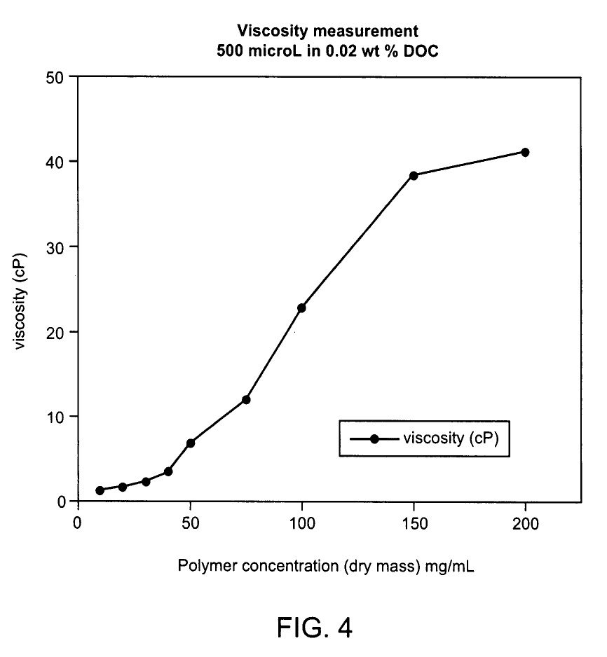

As the concentration of nanoparticles in a gel increases the gel's physical properties change and the viscosity increases. FIG. 4 shows the increase in viscosity that occurs in a shape changing gel as the concentration of pHEMA nanoparticles (dry mass) is increased in a water suspension.

In the above plot, the viscosity increases nearly linearly up to approximately 35 cP at 150 mg/mL dry polymer mass and then levels off at 200 mg/mL dry polymer mass which is near the limit of dispersibility for pHEMA nanoparticles in 0.02 wt % deoxycholate water solution. As the concentration of pHEMA polymer in the gel increases above 50 mg/mL the shear viscosity of the gel increased over time under continuous force.

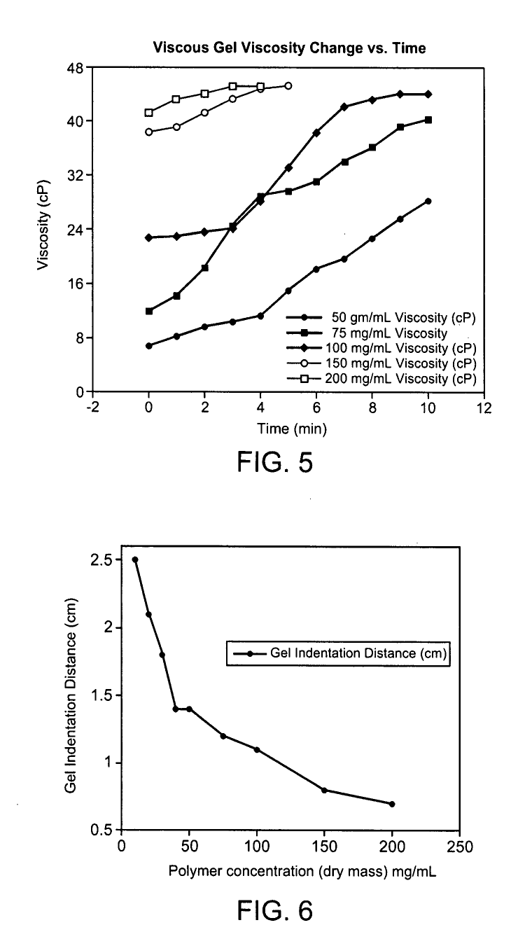

FIG. 5 shows the change in viscosity over time for gels with a concentration of 50 mg/mL of polymer or greater. The data in FIG. 5 shows that the viscosity of the gels increases to a maximum of between 40 and 50 cP in a 10 minute period under shear.

12. Control of Elasticity of Shape Conforming Gels Utilizing Changes in Nanoparticle Composition and Physical Properties

The chemical composition of nanoparticles in lyophilized hydrogel nanoparticle powder can affect the physical properties of the resulting viscous shape conforming gels as shown in Table 7. As the chemical composition is varied, the relative elasticity can be qualitatively measured by determining the distance that a fixed weight impacts a specific mass, volume and shape of gel. For this experiment, a graduated cylinder with a diameter of 2 cm was filled to a volume of 5 mL which contained a viscoelastic gel column of 3.4 cm tall. A 10 g weight was placed on the surface of the gel carefully so that the weight did not touch the sides of the cylinder and the distance that the weight dented into the surface was measured after the system came to rest for 5 minutes. The measurement was taken 5 times and the average was reported in the table below. In all cases, the gel relaxed to the original shape after the weight was removed.

TABLE 7

| | Indentation |
|---|---|
| Sample | distance (cm) |
| pHEMA | 1.4 |
| pHPMA | 0.6 |
| 50:50 pHEMA:HPMA | 0.8 |
| 85:15 pHEMA:HPMA | 1.1 |
| 33:33:33 | 2.3 |
| pHEMA:HPMA:GMA | |
| 90:10 pHEMA:pHPMA | 1.2 |
| 85:15 pHEMA:pHPMA | 0.9 |
| 75:25 pHEMA:pHPMA | 0.7 |
| 50:50 pHEMA:pHPMA | 0.5 |

The above data shows that changing the chemical composition can affect the relative modulus of the gels. As more of a relatively less hydrophilic monomer such as HPMA is added or more pHPMA polymer nanoparticles are added, the gel becomes more resistant to deformation. If a relatively more hydrophilic monomer such as GMA is added the gel becomes softer and easier to deform.

13. Effect of Particle Concentration in the Gel on the Viscoelastic Physical Properties.

The concentration of nanoparticles can affect the physical properties of viscous shape conforming gels. As the nanoparticle concentration is varied, the relative elasticity can be qualitatively measured by determining the distance that a fixed weight impacts a specific mass, volume and shape of gel. For this experiment, a graduated cylinder with a diameter of 2 cm was filled to a volume of 5 mL using several viscous gels composed of different amounts of suspended nanoparticles. The resulting gel contained within the graduate cylinder gave a height of 3.4 cm. A 10 gram weight was then placed on the surface of the gel carefully so that the weight did not touch the sides of the cylinder and the penetration distance of the weight into the surface of the gel was measured five minutes later at after the system came to equilibrium. The measurement was taken 5 times and the average was reported in the table below. In all cases, the gel relaxed to the original shape after the weight was removed.

FIG. 6. shows the relative indentation distance for gels composed of pHEMA nanoparticles with increasing concentration of polymer. As the concentration is increased, the relative distance of indentation decreases for this nanoparticle size range of 120 nm.

14. Effect of Particle Composition on the Rate of Aggregation.

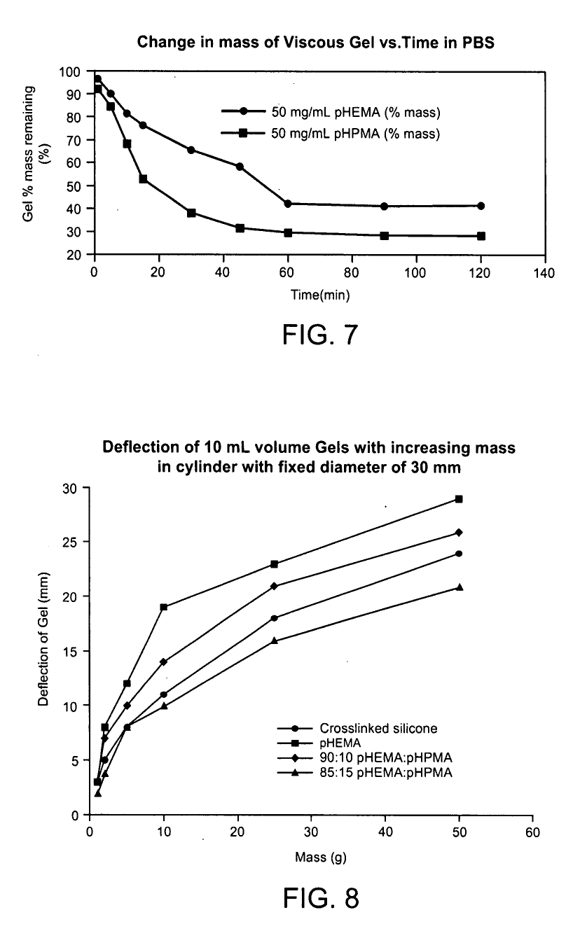

The composition of nanoparticles can affect the extent and rate of aggregation of viscous shape conforming gels when exposed to solutions of physiological ionic strength and pH. Injection of particles into a solution in which the particles have a lower swelling rate, such as a solution of higher ionic strength, forms a hydrogel particle aggregate. The rate of aggregate formation can be quantified by determining the loss of water mass for the gel over time after it is subjected to physiological ionic strength and pH. In a typical experiment, 5 g of a viscous gel suspension of pHEMA or pHPMA nanoparticles at a concentration of 50 mg/mL was added into 100 mL of PBS. The resulting aggregate was allowed to form and was periodically weighed, and returned to the PBS solution. The mass was reported as a percentage of the centrifuged wet polymer mass that shows the amount of water both within and between the particles comprising the aggregate as it collapses. FIG. 7 shows a plot of the rate of aggregation over time from the initial injection to the point at which the aggregate has reached a steady state mass. The plot shows that gels composed of pHEMA particles exhibit a slower aggregation rate and reach a steady state aggregate mass with a higher water composition than corresponding gels composed of pHPMA nanoparticles.

15. Effect of Gel Composition on Indentation.

Powders of different densities and chemical compositions were synthesized, purified and lyophilized. The chemical compositions were:
A. pure pHEMA with 0.01 weight percent sodium deoxycholate salt
B. 90:10 weight:weight ratios of pHEMA:pHPMA with 0.01 weight percent sodium deoxycholate salt
C. 85:15 weight:weight ratios of pHEMA:pHPMA with 0.01 weight percent sodium deoxycholate salt

Studies of the polymers indicate that the relative elastic modulus of a gel formed using the nanoparticle powders can be varied by changing the composition of the nanoparticle powder. For a given concentration of polymer nanoparticles suspended in a shape filling gel without aggregation, the elastic modulus of the resulting gel increases with an increase in the percent composition of pHPMA nanoparticles. The true elastic modulus was not measured for the gels but a deflection of mass was measured in a static cylinder of specific gel volume. Silicone oil was compared as was isolated crosslinked silicone breast implant filler material. Gels contained a 12% weight:volume suspension of polymers in water, while the silicone elastomer was studied as isolated from the implant. 10 mL of each gel was constrained within a cylinder with a fixed diameter of 30 mm. A cup with an exterior diameter of 29 mm was placed on the surface of the gel within the cylinder and the cup mass was varied by adding or subtracting water. The water did not come into contact with the gel.

FIG. 8 shows the results of the indentation study. From the plot, the gels all show a non-linear deflection which is likely because of a combination of both compression and the volume constraints of the cylinder. Although it is difficult to extract the exact elastic modulus from this data, the measurements indicate that increasing the pHPMA nanoparticle percentage in the mixture decreases the amount of deflection. In all cases, removing the mass from the surface resulted in an immediate relaxation. It was hoped that the time component for the relaxation could be estimated for the gels, however, because there was no feedback loop associated with the relaxation in the experiment, a value for tau could not be accurately measured. Qualitative observations indicate that the elastic modulus of the gels increases with increasing percent composition of pHPMA in a mixture. The increase in qualitative elastic modulus is likely a component of the greater hydrophobicity that the hydroxypropylmethacrylate polymer has relative to hydroxyethylmethacrylate.

16. Effect of Gel Composition on Indentation.

Studies indicate that the elastic modulus of the resulting gels can be affected by changing the weight percent of the nanoparticle polymer powder in the gel. The chemical compositions were:
A. pure pHEMA with 0.01 weight percent sodium deoxycholate salt
B. 90:10 weight:weight ratios of pHEMA:pHPMA with 0.01 weight percent sodium deoxycholate salt
C. 85:15 weight:weight ratios of pHEMA:pHPMA with 0.01 weight percent sodium deoxycholate salt

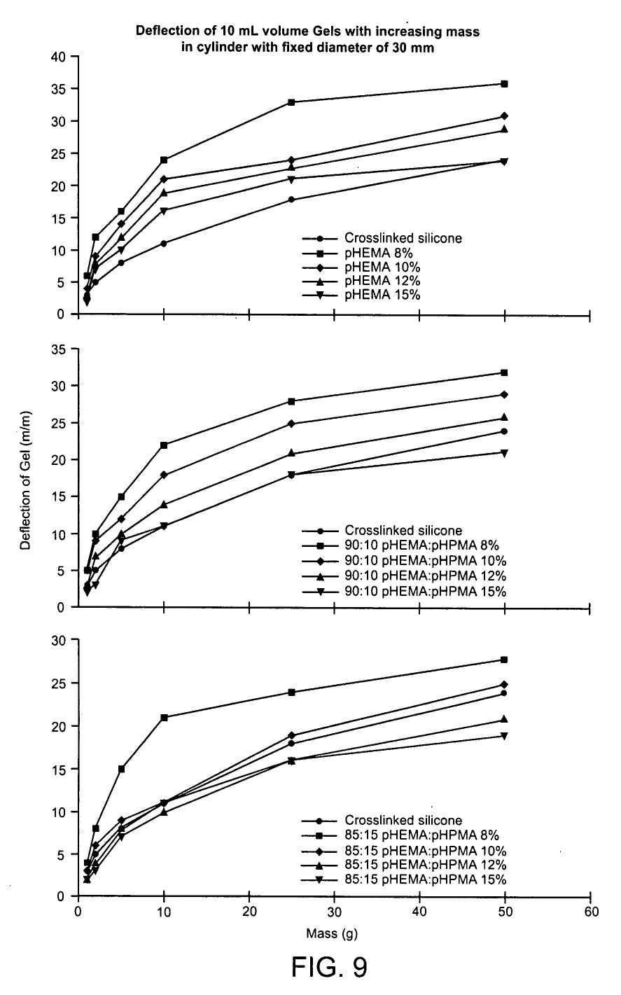

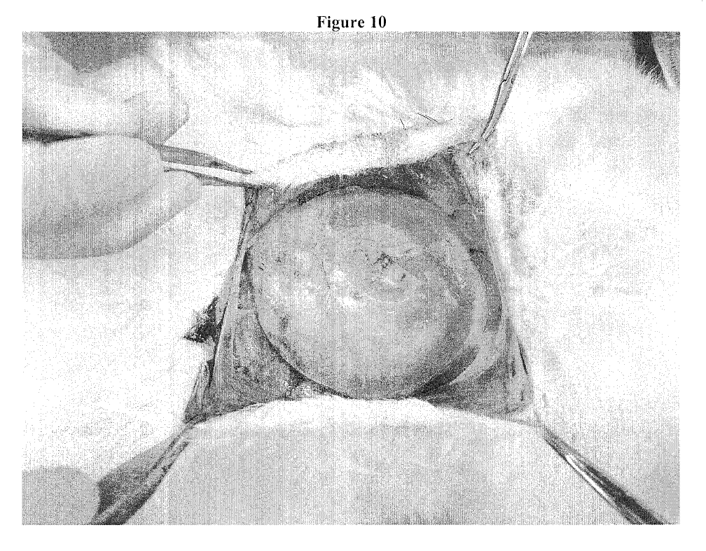

Gels were formed with 8, 10, 12.5 and 15% (weight:volume) suspension of polymers in water, while the silicone elastomer was studied as isolated from the implant. 10 mL of each gel was constrained within a cylinder with a fixed diameter of 30 mm. A cup with an exterior diameter of 29 mm was placed on the surface of the gel within the cylinder and the cup mass was varied by adding or subtracting water. The water did not come into contact with the gel. FIG. 9 shows the deflection of gels of a given composition with different weight percent of the gel in water. The silicone elastomer is shown on each plot as a control. The data indicates that the silicone elastomer gel modulus is best represented using a 15% weight/volume gel composed of 90:10 pHEMA:pHPMA or a 12% weight volume gel composed of 85:15 pHEMA:pHPMA.

17. Filling of Shell with Gel and Rupturing of Shell

A silicone elastomer shell of 200 mL of volume was procured. 200 mL of a 10% pHEMA nanoparticle gel powder mixed in water was added to the shell and the shell was sealed. The gel showed no change in physical properties over a 30 day period. After 30 days, the gel was ruptured in physiological saline where the released gel formed a solid, shape retentive aggregate over a 10 minute period.

18. Filling of Shell with Gel and Rupturing of Shell in an Animal Model

A silicone elastomer shell of 100 mL of volume was procured. 100 mL of a 10% pHEMA containing 0.01% rhodamine methacrylate in the polymer nanoparticle gel powder mixed in water was added to the shell. The shell was implanted in a female New Zealand White rabbit and ruptured. The animal was sacrificed and the aggregate was studied. The aggregate showed no sign of migration and the lung, liver, spleen and lymphatic tissues were free of particles. No loss of aggregate mass was found. FIG. 10 shows the intact aggregate after gross surgical exposure.

Those skilled in the art will recognize that, while specific embodiments and examples have been described, various modifications and changes may be made without departing from the spirit and scope of this invention.

For example, it will be appreciated that this invention relates to a method of formation of viscous, shape conforming gels and their uses as either medicated or unmedicated mammalian implants. The method involves complex interactions of a wide range of factors that may affect the physical characteristics of the viscous, shape conforming gels formed. In addition to those factors expressly discussed herein, other such factors may become apparent to those skilled in the art based on the disclosures herein. The applications of such additional factors of variations in the factors and of combinations of factors are all within the scope of this invention.

Similarly, the methods of this invention will have a vast range of applications. While some applications have been described above, other applications will become apparent to those skilled in the art based on the disclosures herein. All such applications that involve the methods of this invention to form a viscous, shape conforming gel are within the scope of this invention.

1. A method of forming a viscous, shape conforming suspension of gel particles, comprising:
dispersing an effective amount of a dry powder comprising a plurality of gel particles having an average diameter of less than 1 micrometer, wherein the gel particles comprise an effective amount of a plurality of polymeric strands obtained by polymerization of an effective amount of a monomer or two or more monomersat least one of which is selected from the group consisting of a 2-alkenoic acid, a hydroxy (2C-4C) alkyl 2-alkenoate, a dihydroxy (2C-4C) alkyl 2-alkenoate , a hydroxy (2C-4C) alkoxy (2C-4C) alkyl 2-alkenoate, a (1C-4C) alkoxy (2C-4C) alkoxy (2C-4C) alkyl 2-alkenoate or a vicinyl epoxy (1C-4C) alkyl 2-alkenoate, in a polar liquid or a mixture of two or more miscible liquids, at least one of which is polar, and an effective amount of a surfactant to stabilize the plurality of gel particles, thereby forming a suspension of gel particles wherein the particles are concentrated at from about 300 to about 1200 mg wet weight/mL in the suspension system.

2. The method of claim 1, wherein the at least one monomer is acrylic acid, methacrylic acid, 2-hydroxyethyl acrylate, 2-hydroxyethylmethacrylate, diethyleneglycol monoacrylate, diethyleneglycol monomethacrylate, 2-hydroxypropyl acrylate, 2-hydroxypropyl methacrylate, 3-hydroxypropyl acrylate, 3-hydroxypropyl methacrylate, dipropylene glycol monoacrylate, dipropylene glycol monomethacrylate, gylcidyl methacrylate, 2,3-dihydroxypropyl methacrylate, or glycidyl acrylate.

3. The method of claim 1, wherein the monomer(s) is/are 2-hydroxyethyl methacrylate, 2-hydroxypropyl methacrylate, 3-hydroxypropyl methacrylate, 2,3 dihydroxypropyl methacrylate, or a combination thereof.

4. The method of claim 1, wherein the at least one monomer is 2-hydroxyethyl methacrylate, 2-hydroxypropyl methacrylate, 3-hydroxypropyl methacrylate or 2,3-dihydroxypropyl methacrylate.

5. The method of claim 1, wherein the polymer is obtained by polymerization of only one monomer type.

6. The method of claim 5, wherein the one polymer type is 2-hydroxyethyl methacrylate, 2-hydroxypropyl methacrylate, 3-hydroxypropyl methacrylate or 2,3-dihydroxypropyl methacrylate.

7. The method of claim 1, wherein the polymer is obtained by polymerization of 2-hydroxyethyl methacrylate and 2,3-dihydroxypropyl methacrylate.

8. The method of claim 1, wherein the polymer is obtained by polymerization of homopolymers of 2-hydroxyethyl methacrylate and 2,3-dihydroxypropyl methacrylate and blending various ratios.

9. The method of claim 1, wherein the gel particles are about the same average diameter, are formed from one or more monomers and are of a narrow polydispersivity.

10. The method of claim 1, wherein the gel particles are of differing average diameter, are formed from one or more monomers and are of a narrow polydispersivity.

11. The method of claim 1, wherein the gel particles are formed from one or more monomers and are of a broad polydispersivity.

12. The method of claim 1, wherein the plurality of gel particles in the suspension system is at a concentration in the range of 5-20% that results in cluster formation.

13. The method of claim 1, wherein the effective amount of the surfactant is from about 0.005 weight percent to about 0.50 weight percent.

14. The method of claim 1, wherein the average diameter of the gel particles is from about 10 to about 1,000 nanometers.

15. The method of claim 1, wherein the average diameter of the gel particles is from about 40 to about 800 nanometers.

16. The method of claim 1, wherein the gel particles are at a concentration of from about 500 to about 900 mg wet weight/mL in the suspension system.

17. The method of claim 1, wherein the polymeric strands have an average molecular weight of from about 15,000 to about 2,000,000.

18. The method of claim 1, wherein the plurality of polymeric strands are obtained by a process comprising:
i) adding from about 0.01 to about 10 mol percent of a surfactant to a polymerization system comprising a monomer or two or more monomers selected from the group consisting of a 2-alkenoic acid, a hydroxy (2C-4C) alkyl 2-alkenoate, a dihydroxy (2C-4C) alkyl 2-alkenoate , a hydroxy (2C-4C) alkoxy (2C-4C) alkyl 2-alkenoate, a (1C-4C) alkoxy (2C-4C) alkoxy (2C-4C) alkyl 2-alkenoate or a vicinyl epoxy (1C-4C) alkyl 2-alkenoate, and a polar liquid or a mixture of two or more miscible liquids at least one of which is polar liquids, wherein the polar liquid or at least one of the two or more polar liquids comprise(s) one or more hydroxy groups;
ii) polymerizing the monomer(s) to form a plurality of gel particles, each particle comprising a plurality of polymer strands;
iii) isolating the gel particles.

19. The method of claim 1, wherein the liquids are selected from the group consisting of water, a (2C-7C) alcohol, a (3C-8C) polyol and a hydroxy-terminated polyethylene oxide.

20. The method of claim 1, wherein the liquids are selected from the group consisting of water, ethanol, isopropyl alcohol, benzyl alcohol, polyethylene glycol 200-600 and glycerine.

21. The method of claim 18, wherein the liquid is water.

22. The method of claim 18, wherein the method further comprises adding from bout 0.1 to about 15% mol percent of a cross-linking agent to the polymerization system.

23. The method of claim 22, wherein the cross-linking agent is selected from the group consisting of ethylene glycol diacrylate, ethylene glycol dimethacrylate, 1,4-dihydroxybutane dimethacrylate, diethylene glycol dimethacrylate, propylene glycol dimethacrylate, diethylene glycol diacrylate, dipropylene glycol dimethacrylate, dipropylene glycol diacrylate, divinyl benzene, divinyltoluene, diallyl tartrate, diallyl malate, divinyl tartrate, triallyl melamine, N,N′-methylene bisacrylamide, diallyl maleate, divinyl ether, 1,3-diallyl 2-(2-hydroxyethyl) citrate, vinyl allyl citrate, allyl vinyl maleate, diallyl itaconate, di(2-hydroxyethyl) itaconate, divinyl sulfone, hexahydro-1,3,5-triallyltriazine, triallyl phosphite, diallyl benzenephosphonate, triallyl aconitate, divinyl citraconate, trimethylolpropane trimethacrylate and diallyl fumarate.

24. The method of claim 18, wherein step i) of the method further comprises:
adding an effective occluding amount of one or more pharmaceutically active agent(s) to the polar liquid(s) of the polymerization system prior to polymerization or after redispersing the gel particles in the liquid(s).

25. The method of claim 24, wherein the effective amount of the pharmaceutically active agent-containing gel particles occlude from about 0.1 to about 90 weight per cent pharmaceutically active agent-containing liquid.

26. The method of claim 18, wherein the method comprises:
i) adding one or more first pharmaceutically active agent(s) to the polymerization system in an amount effective to give a first pharmaceutically active agent-containing liquid, wherein after polymerization, a portion of the first pharmaceutically active agent-containing liquid is occluded by the gel particles;
ii) isolating the gel particles containing the pharmaceutically active agent(s);
iii) redispersing the gel particles in the polar liquid(s); and
iv) adding one or more second pharmaceutically active agent(s) to the suspension to give a second pharmaceutically active agent-containing liquid, wherein the first pharmaceutically active agent(s) may be the same as or different than the second pharmaceutically active agent(s) and the liquid of the first pharmaceutically active agent-containing liquid may be the same as or different than the liquid of the second pharmaceutically active agent-containing liquid.

27. A viscous, shape conforming gel prepared by the method of claim 1.

28. A medical prosthesis comprising the viscous, shape conforming gel of claim 27.

29. A method for mammalian tissue reconstruction comprising implanting the medical prosthesis of claim 28 in a patient in need thereof.

30. A mammalian tissue reconstruction implant, wherein the mammalian tissue reconstruction implant comprises the viscous, shape conforming gel of claim 29 in a shape adapted for mammalian tissue reconstruction.
